(19) United States
(12) Patent Application Publication
(10) Pub. No.: US 2020/0261596 A1
(43) Pub. Date: Aug. 20, 2020

(54) Therapeutic Particles with Peptide Boronic Acid or Boronate Ester Compounds and Methods of Making and Using Same

(71) Applicant: Pfizer Inc., New York, NY (US)

(72) Inventors: Mir Mukkaram Ali, Woburn, MA (US); Maria Conceicao Figueiredo, Somerville, MA (US); Jeffrey Hrkach, Lexington, MA (US); Young-Ho Song, Natick, MA (US); James Wright, Lexington, MA (US); Stephen E. Zale, Hopkinton, MA (US)

(73) Assignee: Pfizer Inc., New York, NY (US)

(21) Appl. No.: 16/343,023

(22) PCT Filed: Oct. 19, 2017
(86) PCT No.: PCT/IB2017/056518

§ 371 Date: Apr. 18, 2019

Related U.S. Application Data
(60) Provisional application No. 62/410,550, filed on Oct. 20, 2016.

(51) Int. Cl. A61K 47/69; A61K 31/69

(52) U.S. Cl. CPC A61K 47/6937; A61K 9/5161; A61K 31/69

(57) ABSTRACT

Disclosed herein are compositions for treating and preventing diseases such as myeloma and lymphoma, where the compositions include a biocompatible, therapeutic polymeric nanoparticle having a boronate ester compound or a peptide boronic acid compound, and a biodegradable polymer. Methods of use of the therapeutic compositions are also disclosed.

### BACKGROUND

Peptide boronic acid compounds include derivatives of short, e.g., 2-4 amino acid peptides containing aminoboronic acid at the C-terminal end of the peptide sequence. These compounds exhibit a wide range of activities, including inhibition of certain proteolytic enzymes, inhibition of the action of renin, and inhibition of cancer cell growth. In addition, these compounds have been used to reduce the rate of muscle protein degradation, to reduce the activity of NFκB in a cell, to reduce the rate of degradation of p53 protein in a cell, to inhibit cyclin degradation in a cell, to inhibit antigen presentation in a cell, to inhibit NFκB dependent cell adhesion, and to inhibit HIV replication.

Due to the ability to form a stable tetrahedral borate complex between the boronic acid group and the active site serine or histidine moiety, peptide boronic acids are powerful serine-protease inhibitors. This activity is often enhanced and made highly specific towards a particular protease by varying the sequence of the peptide boronic acids and introducing unnatural amino acid residues and other substituents. This optimization led to the selection of peptide boronic acids with powerful antiviral and cytotoxic activities. However, these complexes suffer from the same problems as other short peptides, most notably very fast clearance and limited ability to reach the in vivo target site.

One such peptide boronic compound is bortezomib. Bortezomib is a dipeptide boronic acid derivative and is known as a highly selective, potent, reversible proteasome inhibitor with a Kᵢ of 0.6 nmol/L. Bortezomib has been shown to have activity against a variety of cancer tissues, including breast, ovarian, prostate, lung, and against various tumors, such as pancreatic tumors, lymphomas and melanoma. Bortezomib is typically provided as a mannitol boronic ester, which, in reconstituted form, consists of the mannitol ester in equilibrium with its hydrolysis product, the monomeric boronic acid.

Boronic acid compounds suffer from being rather difficult to obtain in pure form and are susceptible to forming boroxines, which are air-sensitive. Thus, boronic acids are limited as pharmaceutical agents by their complicated characterization and their relatively short shelf life. A need exists for therapeutics that allow for targeted delivery of peptide boronic acids to provide more effective therapy. Derivatization of a peptide boronic acid as its boronate ester may allow such compounds to form part of therapeutic polymeric particles.

### SUMMARY

Provided herein are biocompatible, therapeutic polymeric nanoparticles having a boronate ester compound, or a peptide boronic acid compound such as bortezomib, and a biodegradable or biocompatible polymer such as polylactic acid or polylactic-co-polyglycolic acid, and/or a diblock copolymer such as polylactic acid-co-polyethylene glycol.

In an embodiment, disclosed herein is a biocompatible, therapeutic polymeric nanoparticle comprising a non-esterified boronate compound (e.g., bortezomib); and a biodegradable polymer, such as polyethylene glycol conjugated to polylactic acid or polylactic-co-polyglycolic acid, or a block copolymer comprising a polyethylene portion or block and a block comprising a portion selected from the group consisting of: polylactic acid portion, a polycaprolactone portion, or a polylactic-co-polyglycolic acid portion. In another embodiment, a disclosed therapeutic nanoparticle may include a glyceride such as a monoglyceride, a diglyceride, or a triglyceride. For example, the glyceride may be a monoglyceride (e.g. lauroyl-rac-glycerol). The glyceride may be homogeneously dispersed within the nanoparticle. Such a nanoparticle may comprise, for example, about 0.1 to about 35 percent by weight bortezomib. For example, provided herein is a biocompatible, therapeutic nanoparticle that may include a polylactic acid-polyethylene glycol copolymer where, for example, the polyethylene glycol has an molecular weight of about 4000 to about 6000 g/mol, and/or the polylactic acid has an molecular weight of about 12000 to about 80000 g/mol.

For example, a disclosed nanoparticle may include about 80 to about 90 percent or more by weight polyethylene glycol/polylactic acid copolymer. In another embodiment, a biocompatible, therapeutic nanoparticle is contemplated that includes about 93 to about 98 weight percent mPEG-IPLA and about 1 to about 6 percent by weight bortezomib, wherein the molecular weight of the mPEG is about 5000 Da and the molecular weight of the /PLA is about 16,000 Da.

In another example, a disclosed nanoparticle may include about 10 to about 60 percent or more by weight polyethylene glycol/polylactic acid copolymer or polyethylene glycol/polylactic-co-polyglycolic acid copolylmer and about 5 to about 50 weight percent, or about 10 to about 40 percent or more by weight glyceride (e.g. a monoglyceride, a diglyceride, or a triglyceride). For example, the glyceride may be a monoglyceride (e.g. lauroyl-rac-glycerol). Further, the glyceride may be homogeneously dispersed within the nanoparticle. In an embodiment, a biocompatible, therapeutic nanoparticle is contemplated that includes about 30 to about 40 weight percent PLA/PEG, about 30 to about 40 weight percent lauroyl-rac-glycerol, and about 20 to about 40 weight percent bortezomib, wherein the molecular weight of the PEG is about 5000 Da and the molecular weight of the PLA is about 16,000 Da. In yet another embodiment, therapeutic nanoparticle is contemplated that includes about 30 to about 40 weight percent PLA/PEG, about 30 to about 40 weight percent lauroyl-rac-glycerol, and about 20 to about 40 weight percent bortezomib, wherein the molecular weight of the PEG is about 5000 Da and the molecular weight of the PLA is about 50,000 Da.

In yet another embodiment, a disclosed therapeutic nanoparticle may further include a polylactic acid homopolymer, a polylactic-co-polyglycolic acid homopolymer, and/or a polycaprolactone homopolymer. Such polymers may have for example a carboxcylic or amine end group.

For example, a therapeutic nanoparticle is contemplated that includes about 40 to about 60 weight percent diblock polylactic acid-polyethylene glycol copolymer, about 40 to about 60 weight percent polylactic acid homopolymer or polylactic-co-polyglycolic acid, and about 0.1% to about 15% by weight bortezomib.

Contemplated boronate ester compounds may be formed from a peptide boronic acid compound and a diol, for example, a diol such as a monoglyceride, e.g. 1-undecanoyl-rac-glycerol, monomyristin, monolaurin, and monocaprin, or a biocompatible polymer having a diol functionality, such as a polymer selected from the group consisting of poly(ethyleneglycol)-polydepsipeptide, poly (hydroxypropylmethacrylamide), and poly(methacrylic acid) ester. In some embodiments, the diol may be optionally conjugated to polyethylene glycol. Other diols forming a contemplated boronate ester compound may be selected from the group consisting of 1,2-propanediol, 1,3-propanediol, 1,2-butanediol, 1,3-butanediol, 2,3-butanediol, pinanediol, pinacol, perfluoropinacol, catechol, and 1,2-cyclohexanediol.

For example, a disclosed biocompatible, therapeutic polymeric nanoparticle may include about 20% or about 40% to about 60% by weight polylactic (acid) or polylactic (acid)-polyglycolic acid copolymer; about 40% to about 60% or to about 90% or more by weight polylactic (acid)-polyglycolic acid-polyethylene glycol co-polymer, polylactic (acid)-polyethylene glycol co-polymer or polycaprolactone polyethylene glycol co-polymer; and about 0.1% to about 15% by weight boronic ester compound.

Disclosed nanoparticles, in some embodiments, may include a biodegradable and/or biocompatible polymer and:

wherein

Z is

### or Z₁;

Q is a biocompatible polymer, a polyethylene glycol conjugated lipid, or C₅-C₁₅alkyl,

Z₁ is selected independently for each occurrence, from H and C₁-C₅ alkyl;

Y is a bond or (CH₂)ₙ, where n is 1 or 2; and

R′ is H or C₁-C₃ alkyl.

Also provided herein are therapeutic nanoparticles that include a boronate ester compound such as that formed from a disclosed peptide boronic compound and dextran and/or chitosan optionally conjugated to poly(ethylene) glycol and/or poly (lactic) or poly (lactic)-co-glycolic acid). In an another embodiment, a therapeutic nanoparticle is provided that includes a boronate ester compound such as that formed from poly(lactic) acid conjugated to a mono or di-saccharide.

Also contemplated herein are methods of making disclosed nanoparticles and methods of treating cancers and/or other indications such as multiple myeloma comprising administering to a patient in need thereof a disclosed particle or composition.

### BRIEF DESCRIPTION OF FIGURES

FIG. 1 depicts exemplary peptide boronic acid compounds.

FIG. 2 depicts exemplary boronate ester compounds.

FIG. 3 depicts in-vitro release of three nanoparticle formulations disclosed herein.

FIG. 4 depicts the pharmacokinetic profile of a single dose of bortezomib and a disclosed nanoparticle with bortezomib at 0.5 mg/Kg in Sprague Dawley rats.

FIG. 5 is flow chart for an emulsion process for forming disclosed nanoparticle.

FIG. 6 is a flow diagram for a disclosed emulsion process.

FIG. 7 depicts the effect of feed pressure on resultant particle size.

FIG. 8 depicts in vitro release of bortezomib of various nanoparticles disclosed herein.

FIG. 9 depicts bortezomib loading of various nanoparticles disclosed herein.

FIG. 10 depicts bortezomib loading of various nanoparticles disclosed herein.

FIG. 11 depicts in vitro release of bortezomib of various nanoparticles disclosed herein.

FIG. 12 depicts in vitro release of bortezomib of various nanoparticles disclosed herein.

FIGS. 13A-C depict the pharmacokinetic and tolerability profiles of bortezomib and disclosed nanoparticles with bortezomib. FIG. 13A depicts the pharmacokinetic profile of a single dose of 0.1 mg/Kg bortezomib and disclosed nanoparticles with bortezomib in Sprague Dawley rats. FIGS. 13B and 13C depict the tolerability of bortezomib and disclosed nanoparticles with bortezomib in BalbC mice administered at a dose of 1.0 mg/kg twice a week for three weeks.

FIGS. 14A-C depict the mean tumor volume after administration of bortezomib and disclosed nanoparticles containing bortezomib in an NCI-H460 tumor xenograft mouse model of non-small cell lung cancer. FIGS. 14D-F depict the corresponding tolerability profiles following administration of bortezomib and disclosed nanoparticles containing bortezomib. Bortezomib and disclosed nanoparticles that include bortezomib are administered at 0.5 mg/Kg, 0.75 mg/Kg, or 1.0 mg/Kg in mice twice weekly for three weeks.

FIGS. 15A-B depict the mean tumor volume and tolerability profile after administration of bortezomib and disclosed nanoparticles containing bortezomib in a RPMI-8226 mouse model of multiple myeloma. Bortezomib and disclosed nanoparticles containing bortezomib are administered at 1.0 mg/Kg in mice twice weekly for three weeks.

FIG. 16 A-B depict in vitro release of bortezomib of various nanoparticles disclosed herein.

FIG. 17 depicts in vitro release of bortezomib in various nanoparticles disclosed herein.

FIG. 18 depicts in vitro release of bortezomib in various nanoparticles disclosed herein.

FIG. 19 depicts results of a PK rat study with various rofecoxib nanoparticles disclosed herein.

FIG. 20 A-B depict results of a PK rat study with various bortezomib nanoparticles disclosed herein.

### DETAILED DESCRIPTION

At least in part, this disclosure is directed to particles that include a peptide boronic acid compound (e.g. bortezomib), or a boronate ester compound and, for example, a biodegradable polymer; for example, a therapeutic polymeric nanoparticle.

The features and other details of the disclosure will now be more particularly described. Before further description of the present invention, certain terms employed in the specification, examples and appended claims are collected here. These definitions should be read in light of the remainder of the disclosure and understood as by a person of skill in the art. Unless defined otherwise, all technical and scientific terms used herein have the same meaning as commonly understood by a person of ordinary skill in the art.

### DEFINITIONS

The term “alkoxy” as used herein refers to an alkyl group attached to an oxygen (—O-alkyl-). Exemplary alkoxy groups include, but are not limited to, groups with an alkyl, alkenyl or alkynyl group of 1-12, 1-8, or 1-6 carbon atoms, referred to herein as C₁-C₁₂alkoxy, C₁-C₈alkoxy, and C₁-C₆alkoxy, respectively. Exemplary alkoxy groups include, but are not limited to methoxy, ethoxy, etc. Similarly, exemplary “alkenoxy” groups include, but are not limited to vinyloxy, allyloxy, butenoxy, etc.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀oalkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

Alkyl groups can optionally be substituted with or interrupted by at least one group selected from alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl.

The term “amine” or “amino” as used herein refers to a radical of the form —NR_{d}Rₑ, —N(R_{d})Rₑ—, or —RₑN(R_{d})R_{f}— where R_{d}, Rₑ, and R_{f} are independently selected from alkoxy, alkyl, alkenyl, alkynyl, amide, amino, aryl, arylalkyl, carbamate, cycloalkyl, ester, ether, formyl, halogen, haloalkyl, heteroaryl, heterocyclyl, hydrogen, hydroxyl, ketone, and nitro. The amino can be attached to the parent molecular group through the nitrogen, R_{d}, Rₑ or R_{f}. The amino also may be cyclic, for example any two of Rd, Re or Rf may be joined together or with the N to form a 3- to 12-membered ring, e.g., morpholino or piperidinyl. The term amino also includes the corresponding quaternary ammonium salt of any amino group, e.g., —[N(R_{d})(Rₑ)(R_{f})]⁺. Exemplary amino groups include aminoalkyl groups, wherein at least one of R_{d}, Rₑ, or R_{f} is an alkyl group.

The term “aryl” as used herein refers to refers to a mono-, bi-, or other multi-carbocyclic, aromatic ring system. The aromatic ring may be substituted at one or more ring positions with substituents selected from alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. The term “aryl” also includes polycyclic ring systems having two or more cyclic rings in which two or more carbons are common to two adjoining rings (the rings are “fused rings”) wherein at least one of the rings is aromatic, e.g., the other cyclic rings may be cycloalkyls, cycloalkenyls, cycloalkynyls, and/or aryls. Exemplary aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl.

The term “arylalkyl” or “aralkyl” as used herein refers to an aryl group having at least one alkyl substituent, e.g. -aryl-alkyl-. Exemplary arylalkyl groups include, but are not limited to, arylalkyls having a monocyclic aromatic ring system, wherein the ring comprises 6 carbon atoms. For example, “phenylalkyl” includes phenylC₄alkyl, benzyl, 1-phenylethyl, 2-phenylethyl, etc. Similarly, “aralkoky” as used herein refers to an aryl group having at least one alkoxy substituent, e.g. -aryl-alkoxy-.

The term “carboxy” as used herein refers to the radical —COOH or its corresponding salts, e.g. —COONa, etc.

The term “cycloalkoxy” as used herein refers to a cycloalkyl group attached to an oxygen.

The term “cycloalkyl” as used herein refers to a monovalent saturated or unsaturated cyclic, bicyclic, or bridged bicyclic hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclohexenes, cyclopentanes, cyclopentenes, cyclobutanes and cyclopropanes. Cycloalkyl groups may be substituted with alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. Cycloalkyl groups can be fused to other cycloalkyl, aryl, or heterocyclyl groups.

The terms “halo” or “halogen” or “Hal” as used herein refer to F, Cl, Br, or I.

The terms “heteroaryl” as used herein refers to a 5-15 membered mono-, bi-, or other multi-cyclic, aromatic ring system containing one or more heteroatoms, for example one to four heteroatoms, such as nitrogen, oxygen, and sulfur. Heteroaryls can also be fused to non-aromatic rings. The heteroaryl ring may be substituted at one or more positions with such substituents as described above, as for example, alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl. Illustrative examples of heteroaryl groups include, but are not limited to, acridinyl, benzimidazolyl, benzofuryl, benzothiazolyl, benzothienyl, benzoxazolyl, carbazolyl, carbolinyl, cinnolinyl, furazanyl, furyl, imidazolyl, indazolyl, indolizinyl, indolyl, isobenzofuryl, isoindolyl, isoquinolinyl, isothiazolyl, isoxazolyl, naphthyridinyl, oxadiazolyl, oxazolyl, phenanthridinyl, phenanthrolinyl, phenarsazinyl, phenazinyl, phenothiazinyl, phenoxazinyl, phthalazinyl, pteridinyl, purinyl, pyrazinyl, pyrazolyl, pyrazyl, pyridazinyl, pyridinyl, pyrimidilyl, pyrimidyl, pyrrolyl, quinolinyl, quinolizinyl, quinoxalinyl, quinoxaloyl, quinazolinyl, tetrazolyl, thiadiazolyl, thianthrenyl, thiazolyl, thienyl, thiophenyl, triazinyl, (1,2,3,)- and (1,2,4)-triazolyl, and the like. Exemplary heteroaryl groups include, but are not limited to, a monocyclic aromatic ring, wherein the ring comprises 2 to 5 carbon atoms and 1 to 3 heteroatoms.

The terms “hydroxy” and “hydroxyl” as used herein refers to the radical —OH.

The term “phenyl” as used herein refers to a 6-membered carbocyclic aromatic ring. The phenyl group can also be fused to a cyclohexane or cyclopentane ring. Phenyl can be substituted with one or more substituents including alkanoyl, alkoxy, alkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl and thiocarbonyl.

The term “glycerides” as used herein refers to esters formed from glycerol and fatty acids. Glycerol has three hydroxyl functional groups, which can be esterified with one, two, or three fatty acids. Glycerides can be monoglycerides, diglycerides, and triglycerides.

The term “monoglycerol lipid” or “monoglyceride” as used herein refers to a glyceride consisting of one fatty acid chain covalently bonded to a glycerol molecule through an ester linkage. Monoglycerol lipid can be broadly divided into two groups: 1-monoacylglycerols and 2-monoacylglycerols, depending on the position of the ester bond on the glycerol moiety. Exemplary monoglycerol lipids include, but are not limited to, lauroyl-rac-glycerol, glycerol monomyristate, glycerol monopalmitate, glycerol monostearate, glycerol monoarachidate, glycerol monobehenate, glycerol monopalmitoleate, glycerol monooleate, glycerol monolinoleate, glycerol monolinolenate, glycerol monoarachidonate, and glycerol monocaprylate, and/or for example 1-monomyristoyl-rac glycerol, 1-mono-palmitoyl-rac-glycerol, 2-monopalmitoylglycerol, 1-mono-palmitolenyl-rac-glycerol, 1-monostearoyl-rac-glycerol, 1-monoleoyl-rac-glycerol, 1-monolinoleoyl-rac-glycerol, and 1-monolinolenoyl-rac-glycerol. or combinations thereof.

The term “diglyceride” as used herein refers to a glyceride consisting of two fatty acid chain covalently bonded to a glycerol molecule through an ester linkage. Exemplary diglycerides include, but are not limited to, glycerol dilaurate, glycerol dimyristate, glycerol dipalmitate, glycerol distearate, glycerol diarachidate, glycerol dibehenate, glycerol dipalmitoleate, glycerol dioleate, glycerol dilinoleate, glycerol dilinolenate, glycerol diarachidonate, or combinations thereof.

The term “triglyceride” as used here refers to a glyceride consisting of three fatty acid chain covalently bonded to a glycerol molecule through an ester linkage. Exemplary diglycerides include, but are not limited to, glycerol trilaurate, glycerol trimyristate, glycerol tripalmitate, glycerol tristearate, glycerol triarachidate, glycerol tribehenate, glycerol tripalmitoleate, glycerl trioleate, glycerol trilinoleate, glycerol trilinolenate, glycerol triarachidonate, or combinations thereof.

“Treating” includes any effect, e.g., lessening, reducing, modulating, or eliminating, that results in the improvement of the condition, disease, disorder and the like.

“Pharmaceutically or pharmacologically acceptable” include molecular entities and compositions that do not produce an adverse, allergic or other untoward reaction when administered to an animal, or a human, as appropriate. For human administration, preparations should meet sterility, pyrogenicity, general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

“Individual,” “patient,” or “subject” are used interchangeably and include any animal, including mammals, such as mice, rats, other rodents, rabbits, dogs, cats, swine, cattle, sheep, horses, or primates, and most preferably humans. The compounds and compositions of the invention can be administered to a mammal, such as a human, but can also be other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). “Modulation” includes antagonism (e.g., inhibition), agonism, partial antagonism and/or partial agonism.

In the present specification, the term “therapeutically effective amount” means the amount of the subject compound or composition that will elicit the biological or medical response of a tissue, system, animal or human that is being sought by the researcher, veterinarian, medical doctor or other clinician. The compounds and compositions of the invention are administered in therapeutically effective amounts to treat a disease. Alternatively, a therapeutically effective amount of a compound is the quantity required to achieve a desired therapeutic and/or prophylactic effect.

The term “pharmaceutically acceptable salt(s)” as used herein refers to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. Compounds included in the present compositions that are acidic in nature are capable of forming base salts with various pharmacologically acceptable cations. Examples of such salts include alkali metal or alkaline earth metal salts, such as calcium, magnesium, sodium, lithium, zinc, potassium, and iron salts.

Provided herein, in one embodiment, is a biocompatible, therapeutic polymeric nanoparticle comprising a peptide boronic acid compound (e.g., bortezomib) or a boronate ester compound which may be formed from a peptide boronic acid compound and a diol, and a biodegradable and/or biocompatible polymer such as polylactic acid, polylactic-co-polyglycolic acid, or polycaprolactone, and/or a diblock copolymer of polylactic acid, polylactic-co-polyglycolic acid, or polycaprolactone with polyethylene glycol. In an embodiment, provided herein is a biocompatible, therapeutic polymeric nanoparticle comprising a bortezomib.

### Boronic Acid and Boronate Ester Compounds

Contemplated peptide boronic acid compounds that may form part of the disclosed nanoparticles can include those represented by:

wherein

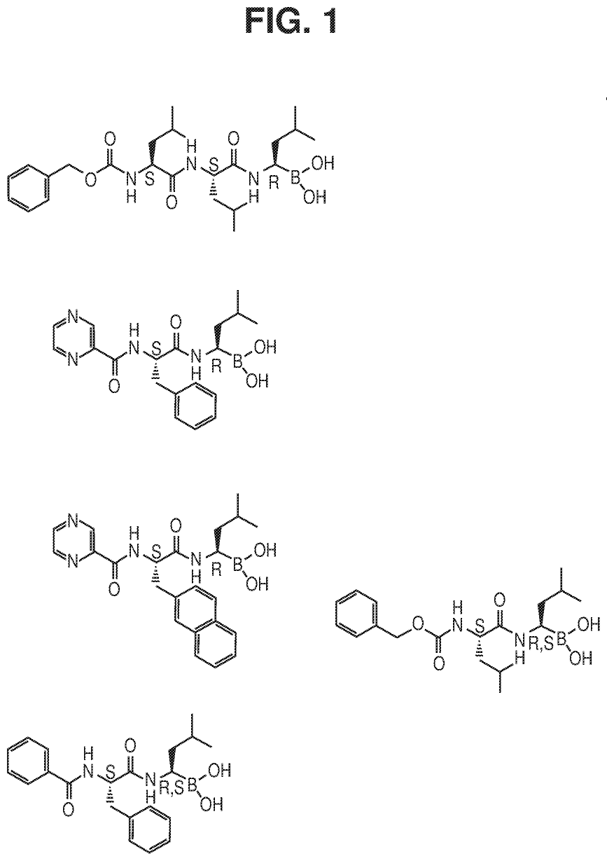

R₁, R₂ and R₃ are independently selected for each occurrence from the group consisting of H, C₁-C₆alkyl, C₁-C₆alkoxy, aryl, aryloxy, aralkyl, aralkoxy, C₃-C₆cycloalkyl, or heterocycle, or any of R₁, R₂ and R₃ can form a heterocyclic ring with an adjacent nitrogen atom in the peptide backbone; and n may be 1, 2, 3, or 4. Exemplary groups for R₁, R₂ and R₃ include, but are not limited to, n-butyl, isobutyl, and neopentyl (alkyl); phenyl or pyrazyl (aryl); 4-((t-butoxycarbonyl)amino)butyl, 3-(nitroamidino)propyl, and (1-cyclopentyl-9-cyano)nonyl (substituted alkyl); naphthylmethyl and benzyl (aralkyl); benzyloxy (aralkoxy); and pyrrolidine (R₂ forms a heterocyclic ring with an adjacent nitrogen atom). Exemplary peptide boronic acids are depicted in FIG. 1. Exemplary peptide boronic acids that may be used include bortezomib (Velcade®), or any of those disclosed in U.S. Publication No. 2006/0159736, U.S. Pat. Nos. 6,083,903, 6,297,217, and 6,617,317, incorporated by reference herein.

Alternatively, boronate ester compounds contemplated herein may be formed from peptide boronic acids such as those of Formula A and a polyol such as a diol. As used herein, a diol refers to a compound having two hydroxyl groups. In some embodiments, a diol may include a monoglyceride, e.g., a diol may be selected from 1-undecanoyl-rac-glycerol, monomyristin, monolaurin, and monocaprin, that may optionally be conjugated to poly(ethylene) glycol. In another embodiment, contemplated diols may include biocompatible polymer having a diol functionality, such as a polymer selected from the group consisting of poly(ethyleneglycol)-polydepsipeptide, poly(hydroxypropylmethacrylamide), and poly(methacrylic acid) ester, for example, contemplated boronate ester compounds may include a peptide boronic acid-biodegradable polymer conjugate.

Also contemplated herein are boronate ester compounds formed from PEGylated lipids based on a lipid moiety (e.g. 10,11 dihydroxyundeconoic acid or 9,10,16-trihydoxyhexadecanoic acid (R,R; R,S; S,S)) that may be conjugated to poly(ethylene) glycol (PEG). For example, a lipid moiety having a diol functionality for boronate ester formation and a reactive moiety for polyethylene glycol may be used for a PEG-lipid-peptide-boronic acid compound, e.g. PEG-lipid-bortezomib.

In an embodiment, boronate ester compounds are contemplated that include boronate ester compounds formed from dextran or chitosan, which may optionally be pegylated as above. For example, boronate ester compounds may be formed from dextran, e.g. having a molecular weight of about 1 kDa, 40 kDa, 60 kDa, 70 kDa, or 300 kDa. Such dextran presents 1,2 and/or 1,3 diols on each repeating unit which each independently may be used to form a boronate ester compound. In some embodiments, a pegylated dextran or chitosan may be used, which may substantially limit or prevent aqueous precipitation of drug at high degrees of e.g. drug-dextran conjugation. For example, pegylated dextran may be used in nanoparticles that may provide similar bortezomib loads to dextran alone.

In some embodiments, boronate ester compounds may be formed from e.g. poly(lactide)-dextran graft. For example, a dextran-graft-PLA-bortezomib may, in some embodiments, have greater hydrophobic character relative to dextran-bortezomib conjugates which may result in more bortezomib accumulation (e.g. greater drug encapsulation) in a nanoparticle core due to its greater hydrophobic character.

Pegylated boronate ester compounds, e.g a PEGylated-lipid-bortezomib compound, may include poly(ethylene) glycol) having a number average molar mass of about 1 to about 10 kDa, e.g., 1 kDa, 2 kDa, 3.5 kDa or 5 kDa, and/or may have a terminus that may be used for e.g. pegylation of a lipid, e.g. an amino terminus.

Also contemplated herein are poly(lactide) or poly(lactic)-co-glycolic polymers conjugated to a mono or di-saccharide reducing sugar, such as D-erythrose, D-threose, D-ribose, D-arabinose, D-Xylose, D-lyxose, D-allose, D-altrose, D-glucose, D-mannose, D-gulose, D-idose, D-galactose, or D-talose, as well as disaccharides such as maltose. Such sugars may be conjugated to amino-terminated poly(lactide) (PLA-NH₂) and used to form boronate ester compounds that may be useful for disclosed nanoparticles.

In other embodiments, boronate ester compounds may be formed from peptide boronic acids such as those of Formula A and a diol selected from the group consisting of 1,2-propanediol, 1,3-propanediol, 1,2-butanediol, 1,3-butanediol, 2,3-butanediol, pinanediol, pinacol, perfluoropinacol, catechol, and 1,2-cyclohexanediol. An exemplary diol is 1,2-propanediol.

In one embodiment, the boronate ester compound may be represented by:

wherein

Z is selected from the group consisting of a biocompatible polymer, a moiety derived from a monoglyceride, and Z₁ wherein Z is optionally substituted by poly(ethylene) glycol, for example, with a polyethylene glycol moiety having a number average molar mass of about 1 to about 10 kDa;

Z₁ is selected independently for each occurrence, from H and C₁-C₅ alkyl;

Y is a bond or (CH₂)ₙ, where n is 1 or 2;

R₁, R₂ and R₃ are independently selected for each occurrence from the group consisting of H, C₁-C₆alkyl, C₁-C₆alkoxy, aryl, aryloxy, aralkyl, aralkoxy, C₃-C₆cycloalkyl, or heterocycle, or any of R₁, R₂ and R₃ can form a heterocyclic ring with an adjacent nitrogen atom in the peptide backbone; and n may be 1, 2, 3, or 4. Exemplary groups for R₁, R₂ and R₃ include, but are not limited to, n-butyl, isobutyl, and neopentyl (alkyl); phenyl or pyrazyl (aryl); 4-((t-butoxycarbonyl)amino)butyl, 3-(nitroamidino)propyl, and (1-cyclopentyl-9-cyano)nonyl (substituted alkyl); naphthylmethyl and benzyl (aralkyl); benzyloxy (aralkoxy); and pyrrolidine (R₂ forms a heterocyclic ring with an adjacent nitrogen atom).

For example, exemplary boronate esters are those depicted in Tables 1 and 2:

TABLE 1

| Diol | X | Y | X′ | Y′ |
|---|---|---|---|---|
| 1,2-Propanediol | Me | H | H | H |
| 1,2-Butanediol | Et | H | H | H |
| 2,3-Butanediol | Me | Me | H | H |
| Pinacol | Me | Me | Me | Me |
| Perfluoropinacol | CF₃ | CF₃ | CF₃ | CF₃ |

```
TABLE 2
  Catechol boronate
  1,2-Cyclohexanediol boronate
  Pinanediol boronate
  X = H: 1,3-Propanediol boronate X = Me: 1,3-Butanediol boronate
```

In another embodiment, the boronate ester compound may be represented by:

wherein

### Z is

Q is a biocompatible polymer or C₅-C₁₅alkyl optionally substituted with for example halo, amino, nitro, or cyano;

Z₁ is selected independently for each occurrence, from H and optionally substituted C₁-C₅ alkyl;

Y is a bond or (CH₂)ₙ, where n is 1 or 2;

and

R′ is H or C₁-C₃alkyl.

Another embodiment provides a biocompatible, therapeutic polymeric nanoparticle comprising:

wherein

Z is

### or Z₁;

Q is a biocompatible polymer or C₅-C₁₅alkyl, wherein Q is optionally substituted with poly(ethylene) glycol;

Z₁ is selected independently for each occurrence, from H and C₁-C₅ alkyl;

Y is a bond or (CH₂)ₙ, where n is 1 or 2; and

R′ is H or C₁-C₃ alkyl.

For example, Q may be a biocompatible polymer comprising poly(methacrylate), poly(2,3-dihydroxypropyl methacrylamide), or poly(ethylene)glycol-poly(depsipeptide). In a further embodiment, Q is C₁₀alkyl. In one embodiment, Q may be a biodegradable polymer, which may be the same or different that the biodegradable polymer forming part of the disclosed nanoparticles. In some embodiments, Q may be selected from and/or comprise any of the polymers discussed below.

Boronate ester compounds contemplated herein also include boronate esters (e.g. bortezomib ester) compounds formed from an alpha-hydroxy carboxylic acid or a beta-hydrooxy carboxylic acid, e.g. from one of the group selected from the group consisting of malic acid, citric acid, 3-hydroxybutyric acid, beta-hydroxyisovaleric acid, tartaric acid, salicylic acid, glucoheptonic acid, maltonic acid, lactobionic acid, galactaric acid, embonic acid, 1-hydroxy-2-naphthoic acid, 3-hydroxy-2-naphthoic acid, and/or pamoic acid or xinafoic acid. For example, contemplated herein are boronate ester compounds represented by:

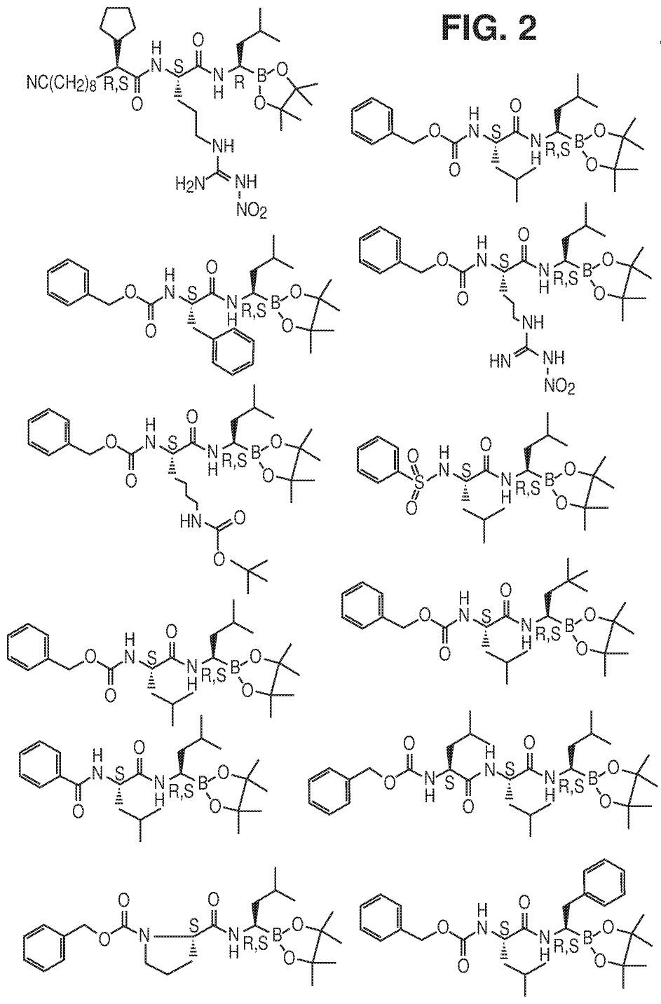

Exemplary boronate ester compounds include those depicted in FIG. 2. Without being limited by any theory, boronic acids and their esters appear to exist in an equilibrium as shown in Scheme A:

For example, compound 1 exists primarily at low pH, whereas increasing pH favors formation of compound 2 which can be trapped as the stable tetrahedral anionic boronate ester 4 by introduction of a diol. The boronate ester 3 can be accessed by minimizing the amount of water present, as water may be necessary to form the neutral trigonal structure. Decreasing the pH leads to dissociation of the boronate ester to the boronic acid.

### Therapeutic Particles

Contemplated biocompatible, therapeutic polymeric nanoparticles include peptide boronic acid or boronate ester compounds such as disclosed above, and a biodegradable polymer and/or biocompatible polymer.

In other embodiments, disclosed therapeutic particles may include a biodegradable polymeric matrix, and/or may be formed from a polymer or lipid (e.g. monoglyceride) conjugated boronate ester compound (e.g. forming a liposome). In one embodiment, the polymeric matrix comprises one, two or more synthetic or natural polymers. The term “polymer,” as used herein, is given its ordinary meaning as used in the art, i.e., a molecular structure comprising one or more repeat units (monomers), connected by covalent bonds. The repeat units may all be identical, or in some cases, there may be more than one type of repeat unit present within the polymer. In some cases, the polymer can be biologically derived, i.e., a biopolymer. Non-limiting examples include peptides or proteins. In some cases, additional moieties may also be present in the polymer, for example biological moieties such as those described below. If more than one type of repeat unit is present within the polymer, then the polymer is said to be a “copolymer.” It is to be understood that in any embodiment employing a polymer, the polymer being employed may be a copolymer in some cases. The repeat units forming the copolymer may be arranged in any fashion. For example, the repeat units may be arranged in a random order, in an alternating order, or as a block copolymer, i.e., comprising one or more regions each comprising a first repeat unit (e.g., a first block), and one or more regions each comprising a second repeat unit (e.g., a second block), etc. Block copolymers may have two (a diblock copolymer), three (a triblock copolymer), or more numbers of distinct blocks.

Disclosed particles can include copolymers, which, in some embodiments, describes two or more polymers (such as those described herein) that have been associated with each other, usually by covalent bonding of the two or more polymers together. Thus, a copolymer may comprise a first polymer and a second polymer, which have been conjugated together to form a block copolymer where the first polymer can be a first block of the block copolymer and the second polymer can be a second block of the block copolymer. Of course, those of ordinary skill in the art will understand that a block copolymer may, in some cases, contain multiple blocks of polymer, and that a “block copolymer,” as used herein, is not limited to only block copolymers having only a single first block and a single second block. For instance, a block copolymer may comprise a first block comprising a first polymer, a second block comprising a second polymer, and a third block comprising a third polymer or the first polymer, etc. In some cases, block copolymers can contain any number of first blocks of a first polymer and second blocks of a second polymer (and in certain cases, third blocks, fourth blocks, etc.). In addition, it should be noted that block copolymers can also be formed, in some instances, from other block copolymers. For example, a first block copolymer may be conjugated to another polymer (which may be a homopolymer, a biopolymer, another block copolymer, etc.), to form a new block copolymer containing multiple types of blocks, and/or to other moieties (e.g., to non-polymeric moieties).

In some embodiments, the polymer (e.g., copolymer, e.g., block copolymer) can be amphiphilic, i.e., having a hydrophilic portion and a hydrophobic portion, or a relatively hydrophilic portion and a relatively hydrophobic portion. A hydrophilic polymer can be one generally that attracts water and a hydrophobic polymer can be one that generally repels water. A hydrophilic or a hydrophobic polymer can be identified, for example, by preparing a sample of the polymer and measuring its contact angle with water (typically, the polymer will have a contact angle of less than 60°, while a hydrophobic polymer will have a contact angle of greater than about 60°). In some cases, the hydrophilicity of two or more polymers may be measured relative to each other, i.e., a first polymer may be more hydrophilic than a second polymer. For instance, the first polymer may have a smaller contact angle than the second polymer.

In one set of embodiments, a polymer (e.g., copolymer, e.g., block copolymer) contemplated herein includes a biocompatible polymer, i.e., the polymer that does not typically induce an adverse response when inserted or injected into a living subject, for example, without significant inflammation and/or acute rejection of the polymer by the immune system, for instance, via a T-cell response. Accordingly, the therapeutic particles contemplated herein can be non-immunogenic. The term non-immunogenic as used herein refers to endogenous growth factor in its native state which normally elicits no, or only minimal levels of, circulating antibodies, T-cells, or reactive immune cells, and which normally does not elicit in the individual an immune response against itself.

Biocompatibility typically refers to the acute rejection of material by at least a portion of the immune system, i.e., a nonbiocompatible material implanted into a subject provokes an immune response in the subject that can be severe enough such that the rejection of the material by the immune system cannot be adequately controlled, and often is of a degree such that the material must be removed from the subject. One simple test to determine biocompatibility can be to expose a polymer to cells in vitro; biocompatible polymers are polymers that typically will not result in significant cell death at moderate concentrations, e.g., at concentrations of 50 micrograms/10⁶ cells. For instance, a biocompatible polymer may cause less than about 20% cell death when exposed to cells such as fibroblasts or epithelial cells, even if phagocytosed or otherwise uptaken by such cells. Non-limiting examples of biocompatible polymers that may be useful in various embodiments of the present invention include polydioxanone (PDO), polyhydroxyalkanoate, polyhydroxybutyrate, poly(glycerol sebacate), polyglycolide, polylactide, PLGA, polycaprolactone, or copolymers or derivatives including these and/or other polymers.

In certain embodiments, contemplated biocompatible polymers may be biodegradable, i.e., the polymer is able to degrade, chemically and/or biologically, within a physiological environment, such as within the body. As used herein, “biodegradable” polymers are those that, when introduced into cells, are broken down by the cellular machinery (biologically degradable) and/or by a chemical process, such as hydrolysis, (chemically degradable) into components that the cells can either reuse or dispose of without significant toxic effect on the cells. In one embodiment, the biodegradable polymer and their degradation byproducts can be biocompatible.

For instance, a contemplated polymer may be one that hydrolyzes spontaneously upon exposure to water (e.g., within a subject), the polymer may degrade upon exposure to heat (e.g., at temperatures of about 37° C.). Degradation of a polymer may occur at varying rates, depending on the polymer or copolymer used. For example, the half-life of the polymer (the time at which 50% of the polymer can be degraded into monomers and/or other nonpolymeric moieties) may be on the order of days, weeks, months, or years, depending on the polymer. The polymers may be biologically degraded, e.g., by enzymatic activity or cellular machinery, in some cases, for example, through exposure to a lysozyme (e.g., having relatively low pH). In some cases, the polymers may be broken down into monomers and/or other nonpolymeric moieties that cells can either reuse or dispose of without significant toxic effect on the cells (for example, polylactide may be hydrolyzed to form lactic acid, polyglycolide may be hydrolyzed to form glycolic acid, etc.).

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids or polyanhydrides.

In other embodiments, contemplated polyesters for use in disclosed nanoparticles may be diblock copolymers, e.g., PEGylated polymers and copolymers (containing poly(ethylene glycol) repeat units) such as of lactide and glycolide (e.g., PEGylated PLA, PEGylated PGA, PEGylated PLGA), PEGylated poly(caprolactone), and derivatives thereof. For example, a “PEGylated” polymer may assist in the control of inflammation and/or immunogenicity (i.e., the ability to provoke an immune response) and/or lower the rate of clearance from the circulatory system via the reticuloendothelial system (RES), due to the presence of the poly(ethylene glycol) groups.

PEGylation may also be used, in some cases, to decrease charge interaction between a polymer and a biological moiety, e.g., by creating a hydrophilic layer on the surface of the polymer, which may shield the polymer from interacting with the biological moiety. In some cases, the addition of poly(ethylene glycol) repeat units may increase plasma half-life of the polymer (e.g., copolymer, e.g., block copolymer), for instance, by decreasing the uptake of the polymer by the phagocytic system while decreasing transfection/uptake efficiency by cells. Those of ordinary skill in the art will know of methods and techniques for PEGylating a polymer, for example, by using EDC (I-ethyl-3-(3-dimethylaminopropyl) carbodiimide hydrochloride) and NHS (N-hydroxysuccinimide) to react a polymer to a PEG group terminating in an amine, by ring opening polymerization techniques (ROMP), or the like.

Other contemplated polymers that may form part of a disclosed nanoparticle may include poly(ortho ester) PEGylated poly(ortho ester), polylysine, PEGylated polylysine, poly(ethylene imine), PEGylated poly(ethylene imine), poly(L-lactide-co-L-lysine), poly(serine ester), poly(4-hydroxy-L-proline ester), poly[α-(4-aminobutyl)-L-glycolic acid], and derivatives thereof. In other embodiments, polymers can be degradable polyesters bearing cationic side chains. Examples of these polyesters include poly(L-lactide-co-L-lysine), poly(serine ester), poly(4-hydroxy-L-proline ester).

In other embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, amino alkyl methacrylate copolymer, poly(acrylic acid), poly(methacrylic acid), methacrylic acid alkylamide copolymer, poly(methyl methacrylate), poly(methacrylic acid polyacrylamide, amino alkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

PLGA contemplated for use as described herein can be characterized by a lactic acid:glycolic acid ratio of e.g., approximately 85:15, approximately 75:25, approximately 60:40, approximately 50:50, approximately 40:60, approximately 25:75, or approximately 15:85. In some embodiments, the ratio of lactic acid to glycolic acid monomers in the polymer of the particle (e.g., a PLGA block copolymer or PLGA-PEG block copolymer), may be selected to optimize for various parameters such as water uptake, therapeutic agent release and/or polymer degradation kinetics can be optimized. In other embodiments, the end group of a PLA polymer chain may be a carboxylic acid group, an amine group, or a capped end group with e.g., a long chain alkyl group or cholesterol.

Particles disclosed herein may or may not contain PEG. In addition, certain embodiments can be directed towards copolymers containing poly(ester-ether)s, e.g., polymers having repeat units joined by ester bonds (e.g., R—C(O)—O—R′ bonds) and/or ether bonds (e.g., R—O—R′ bonds). Contemplated herein in certain embodiments is a biodegradable polymer, such as a hydrolyzable polymer containing carboxylic acid groups, that may be conjugated with poly(ethylene glycol) repeat units to form a poly(ester-ether).

In one embodiment, the molecular weight of the polymers can be optimized for effective treatment as disclosed herein. For example, the weight of a polymer may influence particle degradation rate (such as when the molecular weight of a biodegradable polymer can be adjusted), solubility, water uptake, and drug release kinetics. For example, the molecular weight of the polymer can be adjusted such that the particle biodegrades in the subject being treated within a reasonable period of time (ranging from a few hours to 1-2 weeks, 3-4 weeks, 5-6 weeks, 7-8 weeks, etc.) In an embodiment, a disclosed particle may comprise a copolymer of PEG and PLA, wherein the PEG portion may have a molecular weight of 1,000-20,000 g/mol, e.g., 5,000-20,000, e.g., 4,000-10,000 g/mol, and the PLA portion may have a molecular weight (for example, number average or weight average) of 5,000-100,000 g/mol, e.g., 10,000-80,000, e.g., 14,000-18,000 g/mol).

For example, disclosed biocompatible, therapeutic polymeric nanoparticle may include polylactic (acid)-polyethylene glycol co-polymer and/or polylactic (acid). Alternatively, a disclosed biocompatible, therapeutic polymeric nanoparticle may include polylactic-co-polyglycolic (acid)-polyethylene glycol co-polymer and/or polylactic-co-polyglycolic acid, or polycaprolactone and/or polycaprolactone-co-polyethylene glycol. For example, a disclosed biocompatible, therapeutic polymeric nanoparticle may include about 40% to about 60% by weight polylactic (acid) or polylactic (acid)-polyglycolic acid copolymer; about 40% to about 60% by weight polylactic (acid)-polyglycolic acid-polyethylene glycol co-polymer or polylactic (acid)-polyethylene glycol co-polymer; and about 0.1% to about 15% by weight, or about 0.1% to about 25%, or 0.1% to about 50% by weight boronic ester or bortezomib (e.g. free, non-esterified) compound. In an exemplary embodiment, the particles may include about 90% to about 99% PLA-PEG block copolymer (e.g., mPEG (5,000 Da)-/PLA (16,000 Da)) and about 0.1% to about 10% boronate compound such as bortezomib. In another embodiment, the particle may include about 90% to about 99% PLA-PEG block copolymer (e.g., PEG (5,000 Da)-PLA (50,000 Da)) and about 0.1% to about 10% boronate compound such as bortezomib.

In an embodiment, a biocompatible, therapeutic polymeric nanoparticle contemplated herein may include a substantially hydrophobic boronate ester or boronate compound such as bortezomib, and a PLA-PEG block copolymer or a PLGA-PEG block copolymer. In another embodiment a biocompatible, therapeutic polymeric nanoparticle contemplated herein may further include a glyceride such as a monoglyceride, a diglyceride, or a triglyceride. In an embodiment, the glyceride is not conjugated to PEG to form a boronate ester compound. The glyceride may be homogenously dispersed within the nanoparticle.

In an exemplary embodiment, the particles may include about 30% to about 40% by weight PLA-PEG block copolymer (e.g., PEG (5,000 Da)/PLA (16,000 Da)), about 10% to about 40% by weight glyceride, or about 20% to about 50% by weight glyceride, and about 20% to about 40% by weight boronate compound such as bortezomib. In another embodiment, the particles may include about 30% to about 40% by weight PLA-PEG block copolymer (e.g., PEG (5,000 Da)/PLA (50,000 Da)), about 30% to about 40% by weight glyceride, and about 20% to about 40% by weight boronate compound such as bortezomib.

In general, any glyceride known in the art can be used in the invention. Contemplated glycerides include monoglycerides, diglycerides, and triglycerides.

Exemplary monoglycerides include, but are not limited to, lauroyl-rac-glycerol, glycerol monomyristate, glycerol monopalmitate, glycerol monostearate, glycerol monoarachidate, glycerol monobehenate, glycerol monopalmitoleate, glycerol monopalmitoleate, glycerol monooleate, glycerol monolinoleate, glycerol monolinolenate, glycerol monoarachidonate, glycerol monocaprylate, or combinations thereof.

Exemplary diglycerides include, but are not limited to, glycerol dilaurate, glycerol dimyristate, glycerol dipalmitate, glycerol distearate, glycerol diarachidate, glycerol dibehenate, glycerol dipalmitoleate, glycerl dioleate, glycerol dilinoleate, glycerol dilinolenate, glycerol diarachidonate, or combinations thereof.

Exemplary triglycerides include, but are not limited to, glycerol trilaurate, glycerol trimyristate, glycerol tripalmitate, glycerol tristearate, glycerol triarachidate, glycerol tribehenate, glycerol tripalmitoleate, glycerl trioleate, glycerol trilinoleate, glycerol trilinolenate, glycerol triarachidonate, or combinations thereof.

In an exemplary embodiment, a biocompatible, therapeutic polymeric nanoparticle contemplated herein may include a substantially hydrophobic boronate ester or boronate compound such as bortezomib, and polylactic acid or polylactic-co-polyglycolic acid. In some embodiments, a biocompatible, therapeutic polymeric nanoparticle may further include a targeting ligand. For example, a contemplated boronate ester compound may be formed from a peptide boronic acid compound and a diol selected so as to increase the hydrophobicity of the boronate ester compound. In some embodiments, a more hydrophobic boronate ester compound may be easier to encapsulate in a biodegradable and/or substantially hydrophobic polymer such as PL(G)A and may be less likely to diffuse out of the particle during particle formation and recovery, and/or after the particles have been resuspended in an aqueous medium and/or upon administration to a patient, e.g. by injection.

In some embodiments, disclosed therapeutic particles and/or compositions include targeting agents such as dyes, for example Evans blue dye. Such dyes may be bound to or associated with a therapeutic particle, or disclosed compositions may include such dyes. For example, Evans blue dye may be used, which may bind or associate with albumin, e.g. plasma albumin.

Disclosed therapeutic particles, may, some embodiments, include a targeting moiety, i.e., a moiety able to bind to or otherwise associate with a biological entity. The term “bind” or “binding,” as used herein, refers to the interaction between a corresponding pair of molecules or portions thereof that exhibit mutual affinity or binding capacity, typically due to specific or non-specific binding or interaction, including, but not limited to, biochemical, physiological, and/or chemical interactions. Therapeutic compositions disclosed herein may, for example, be locally administered to a designated region such as a blood vessel.

In certain embodiments, one or more polymers of a disclosed particle may be conjugated to a lipid. The polymer may be, for example, a lipid-terminated PEG. As described below, the lipid portion of the polymer can be used for self assembly with another polymer, facilitating the formation of a particle. For example, a hydrophilic polymer could be conjugated to a lipid that will self assemble with a hydrophobic polymer.

In some embodiments, lipids can be oils. In general, any oil known in the art can be conjugated to the polymers used in the invention. In some embodiments, an oil may comprise one or more fatty acid groups or salts thereof. In some embodiments, a fatty acid group may comprise digestible, long chain (e.g., C₈-C₅₀), substituted or unsubstituted hydrocarbons. In some embodiments, a fatty acid group may be a C₁₀-C₂₀ fatty acid or salt thereof. In some embodiments, a fatty acid group may be a C₁₅-C₂₀ fatty acid or salt thereof. In some embodiments, a fatty acid may be unsaturated. In some embodiments, a fatty acid group may be monounsaturated. In some embodiments, a fatty acid group may be polyunsaturated. In some embodiments, a double bond of an unsaturated fatty acid group may be in the cis conformation. In some embodiments, a double bond of an unsaturated fatty acid may be in the trans conformation.

In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linolenic, gamma-linoleic, arachidonic, gadoleic, arachidonic, eicosapentaenoic, docosahexaenoic, or erucic acid.

In one embodiment, the lipid can be of the Formula V:

and salts thereof, wherein each R is, independently, C₁₋₃₀ alkyl. In one embodiment of Formula V, the lipid can be 1,2 distearoyl-sn-glycero-3-phosphoethanolamine (DSPE), and salts thereof, e.g., the sodium salt.

In another embodiment, a disclosed particle can be associated with (e.g., surrounded by) a small molecule amphiphilic compound e.g. having as possible components: 1) a biodegradable polymeric material that forms the core of the particle, which can carry bioactive drugs and release them at a sustained rate after cutaneous, intravenous, subcutaneous, mucosal, intramuscular, ocular, systemic, oral or pulmonary administration; 2) a small molecule amphiphilic compound that surrounds the polymeric material forming a shell for the particle; and optionally 3) a targeting molecule that can bind to a unique molecular signature on cells, tissues, or organs of the body.

Also provided herein are particles that include a lipid (e.g. monoglyceride), dextran or chitosan that may form part of a boronate ester forming component and also may be capable of forming a therapeutic particle (which then may or may not include a biodegradable polymer). For example, provided herein is a poly(ethylene)glycol-lipid conjugated to a peptide boronic compound to form a boronate ester compound. Also provided herein are poly(ethylene)glycol conjugated dextran and/or chitosan compound conjugated to peptide boronic compounds. Such pegylated compounds may act without substantially triggering the immune system of a patient after administration (e.g. by i.v.) for a period of time (e.g. protecting them from detection and clearance) so that an effective amount of the peptide boronic acid can be delivered. Such particles may, for example, partition into the leaky vasculature of solid tumors leading to drug accumulation in the tumor site with enhanced permeability and retention effect).

In some embodiments, colloidal suspensions, e.g. aqueous colloidal suspensions, are provided that include a lipid- peptide boronic acid conjugate, wherein the lipid is optionally conjugated to poly(ethylene) glycol. Contemplated suspensions include those having micellar and small unilamellar vesicles (about 15-30 nm); large unilamellar vesicles large unilamellar vesicles (about 100-200 nm) and/or liposomes (about 100-500 nm). Such suspensions can be prepared by well known methods including sonication, extrusion, dialysis and hydration of lipid monolayers.

In some embodiments, a lipid (e.g. monoglyceride) moiety may provide protection of the boronate ester compound from hydrolysis which may control release of e.g., bortezomib from particles that include a boronate ester compound formed from a lipid. The lipid based nanoparticle suspension will be stored as dry lyophilized powder and re-suspended immediately prior to use. Alternatively, such nanoparticle suspension may be stored frozen and thawed immediately prior to use.

Disclosed PEGylated-lipid-bortezomib conjugates described above may also be used in a hybrid polymer-lipid based nanoparticle system. For example, in some embodiments, the molar mass of biodegradable polymers may be about 5 kDa to 100 kDa. Diblock copolymer will be based on PEG of molar masses 2 kDa, 3.5 kDa, 5 kDa, and 10 kDa and poly(lactide) or poly(lactide-co-glycolide) of molar mass between 5 kDa and 50 kDa.

Also disclosed herein are compositions comprising a plurality of biocompatible, therapeutic polymeric nanoparticles as disclosed herein and a pharmaceutically acceptable excipient. Nanoparticles as disclosed herein have a characteristic dimension of less than about 1 micrometer, where the characteristic dimension of a particle is the diameter of a perfect sphere having the same volume as the particle. For example, a particle may have a characteristic dimension of the particle that may be less than about 300 nm, less than about 200 nm, less than about 150 nm, less than about 100 nm, less than about 50 nm, less than about 30 nm, less than about 10 nm, less than about 3 nm, or less than about 1 nm in some cases. In some embodiments, a disclosed particle may have a diameter of 50 nm-200 nm.

In general, the particles disclosed herein can be about 40 nm to about 500 nm in size, for example, may be less than or equal to about 90 nm in size, e.g., about 40 nm to about 80 nm, e.g., about 40 nm to about 60 nm. For example, particles less than about 90 nm in size, may reduce liver uptake by the subject, and may thereby allow longer circulation in the bloodstream.

In an embodiment, particles disclosed herein may have a surface zeta potential ranging from about −80 mV to 50 mV. Zeta potential is a measurement of surface potential of a particle. In some embodiments, the particles can have a zeta potential ranging between 0 mV and −50 mV, e.g., between −1 mV and 50 mV. In some embodiments, the particles can have a zeta potential ranging between −1 mV and −25 mV. In some embodiments, the particles can have a zeta potential ranging between −1.1 mV and −10 mV.

In some embodiments, when exposed to aqueous suspension conditions, the core of a disclosed nanoparticle may hydrate, leading to dissociation of the boronate ester or peptide boronic acid compound. Release of the resulting boronic acid, may, in some embodiments, be modulated by the polymer nanoparticle forming polymers and the structural aspects of the drug-polymer conjugate. In some aspects, the boronic acid is dissociated in a controlled release manner. In other embodiments, the boronic acid release is localized as a result of a targeting moiety in or on the nanoparticle

### Methods of Making Compounds and Nanoparticles

Contemplated herein are methods of reacting a boronic acid drug with a diol to form a boronic ester that is suitable for encapsulation in a polymeric matrix and formation of biocompatible, therapeutic polymeric nanoparticles. For example, a disclosed boronate ester compound may increase the hydrophobicity of the compound, such that it is easier to encapsulate and less likely to diffuse from the nanoparticle during formation and delivery. In some embodiments, formation of the nanoparticles allows for encapsulation of the boronate ester prior to its dissociation to the boronic acid. In other embodiments, formation of the nanoparticles allows for the boronate ester to fully form prior to administration. In another embodiment, the boronic acid may be reacted with a diol, and then the reaction mixture is combined with a polymer solution in a one-step process. Nano-emulsion of the mixture then provides the therapeutic nanoparticles.

For example, provided herein is a method of preparing a plurality of biocompatible, therapeutic polymeric nanoparticles comprising: combining a boronate ester compound and a biodegradable polymer with an organic solution to form a first organic phase; combining the first organic phase with a first aqueous solution to form a second phase; emulsifying the second phase to form an emulsion phase; adding a drug solubilizer to the emulsion phase to form a solubilized phase; and recovering the biocompatible, therapeutic polymeric nanoparticles.

In yet another embodiment, provided herein is a method of preparing a plurality of biocompatible, therapeutic polymeric nanoparticles comprising: combining a peptide boronic acid compound (e.g. bortezomib), a biodegradable polymer, and a glyceride (e.g. a monoglyceride, a diglyceride, or a triglyceride) with an organic solution to form a first organic phase; combining the first organic phase with a first aqueous solution to form a second phase; emulsifying the second phase to form an emulsion phase; adding a drug solubilizer to the emulsion phase to form a solubilized phase; and recovering the biocompatible, therapeutic polymeric nanoparticles. In an embodiment, the glyceride is a monoglyceride (e.g. lauroyl-rac-glycerol). The glyceride may be homogenously dispersed within the nanoparticle.

Another embodiment provides a method of preparing a plurality of biocompatible, therapeutic polymeric nanoparticles comprising: contacting a peptide boronic acid compound and a diol to form a reaction mixture comprising a boronate ester compound; combining the reaction mixture and a biodegradable polymer with an organic solution to form a first organic phase; combining the first organic phase with a first aqueous solution to form a second phase; emulsifying the second phase to form an emulsion phase; adding a drug solubilizer to the emulsion phase to form a solubilized phase; and recovering the biocompatible, therapeutic polymeric nanoparticles.

Also provided herein is a method of preparing a plurality of biocompatible, therapeutic polymeric nanoparticles comprising: combining a peptide boronic acid compound and a biodegradable polymer with an organic solvent to form a first organic phase; combining the first organic phase with a first aqueous solution to form a second phase; emulsifying the second phase to form an emulsion phase; adding a drug solubilizer to the emulsion phase to form a solubilized phase; recovering nanoparticles; and contacting the nanoparticles or the emulsion phase with a diol to form biocompatible, therapeutic polymeric nanoparticles that include a boronate ester compound.

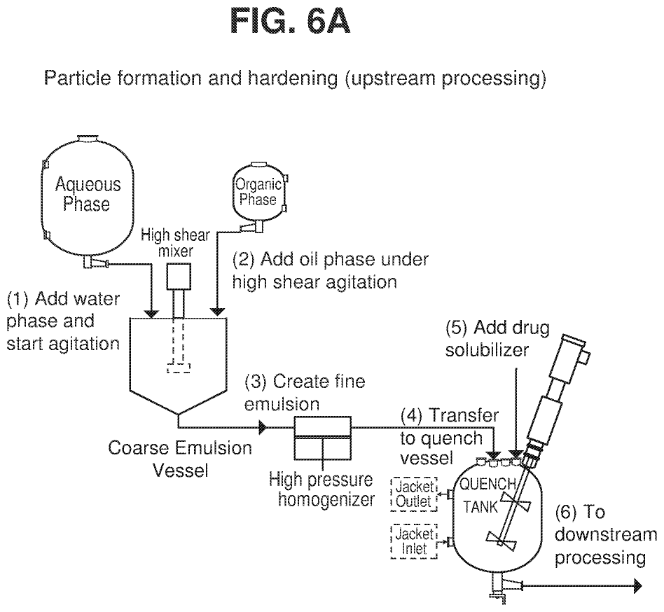

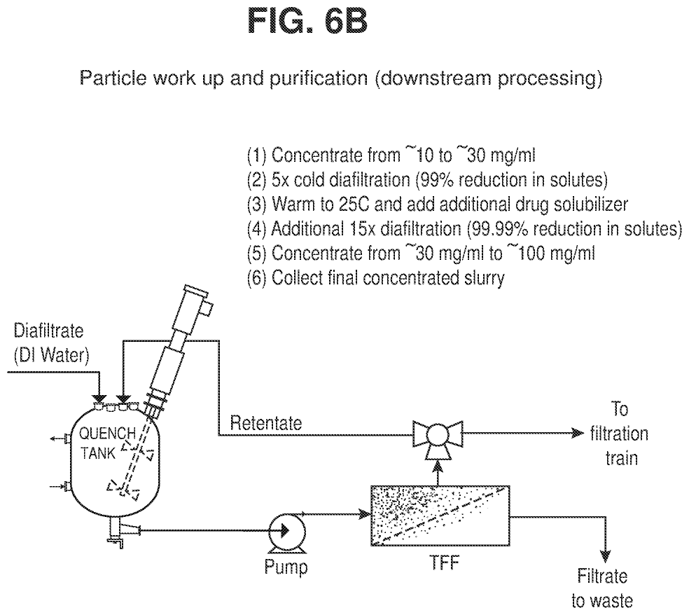

In an embodiment, a nanoemulsion process is provided, such as the process represented in FIGS. 5 and 6. For example, a therapeutic agent such as bortezomib, a first polymer (for example, PLA-PEG or PLGA-PEG) and a second polymer (e.g. (PL(G)A or PLA), with an organic solution to form a first organic phase. Such first phase may include about 5 to about 50% weight solids, e.g about 5 to about 40% solids, or about 10 to about 30% solids, e.g. about 10%, 15%, 20% solids. The first organic phase may be combined with a first aqueous solution to form a second phase. The organic solution can include, for example, acetonitrile, tetrahydrofuran, ethyl acetate, isopropyl alcohol, isopropyl acetate, dimethylformamide, methylene chloride, dichloromethane, chloroform, acetone, benzyl alcohol, Tween 80, Span 80,or the like, and combinations thereof. In an embodiment, the organic phase may include benzyl alcohol, ethyl acetate, and combinations thereof. The second phase can be between about 1 and 50 weight %, e.g., 5-40 weight %, solids. The aqueous solution can be water, optionally in combination with one or more of sodium cholate, ethyl acetate, and benzyl alcohol.

For example, the oil or organic phase may use a solvent that is only partially miscible with the nonsolvent (water). Therefore, when mixed at a low enough ratio and/or when using water pre-saturated with the organic solvents, the oil phase remains liquid. The oil phase may bee emulsified into an aqueous solution and, as liquid droplets, sheared into nanoparticles using, for example, high energy dispersion systems, such as homogenizers or sonicators. The aqueous portion of the emulsion, otherwise known as the “water phase”, may be surfactant solution consisting of sodium cholate and pre-saturated with ethyl acetate and benzyl alcohol.

Emulsifying the second phase to form an emulsion phase may be performed in one or two emulsification steps. For example, a primary emulsion may be prepared, and then emulsified to form a fine emulsion. The primary emulsion can be formed, for example, using simple mixing, a high pressure homogenizer, probe sonicator, stir bar, or a rotor stator homogenizer. The primary emulsion may be formed into a fine emulsion through the use of e.g. probe sonicator or a high pressure homogenizer, e.g. by using 1, 2, 3 or more passes through a homogenizer. For example, when a high pressure homogenizer is used, the pressure used may be about 4000 to about 8000 psi, or about 4000 to about 5000 psi, e.g. 4000 or 5000 psi.

Either solvent evaporation or dilution may be needed to complete the extraction of the solvent and solidify the particles. For better control over the kinetics of extraction and a more scalable process, a solvent dilution via aqueous quench may be used. For example, the emulsion can be diluted into cold water to a concentration sufficient to dissolve all of the organic solvent to form a quenched phase. Quenching may be performed at least partially at a temperature of about 5° C. or less. For example, water used in the quenching may be at a temperature that is less that room temperature (e.g. about 0 to about 10° C., or about 0 to about 5° C.).

In some embodiments, not all of the therapeutic agent, e.g. bortezomib, is encapsulated in the particles at this stage, and a drug solubilizer is added to the quenched phase to form a solubilized phase. The drug solubilizer may be for example, Tween 80, Tween 20, polyvinyl pyrrolidone, cyclodextran, sodium dodecyl sulfate, or sodium cholate. For example, Tween-80 may added to the quenched nanoparticle suspension to solubilize the free drug and prevent the formation of drug crystals. In some embodiments, a ratio of drug solubilizer to therapeutic agent is about 100:1 to about 10:1.

The solubilized phase may be filtered to recover the nanoparticles. For example, ultrafiltration membranes may be used to concentrate the nanoparticle suspension and substantially eliminate organic solvent, free drug, and other processing aids (surfactants). Exemplary filtration may be performed using a tangential flow filtration system. For example, by using a membrane with a pore size suitable to retain nanoparticles while allowing solutes, micelles, and organic solvent to pass, nanoparticles can be selectively separated. Exemplary membranes with molecular weight cut-offs of about 300-500 kDa (˜5-25 nm) may be used.

Diafiltration may be performed using a constant volume approach, meaning the diafiltrate (cold deionized water, e.g. about 0° C. to about 5° C., or 0 to about 10° C.) may added to the feed suspension at the same rate as the filtrate is removed from the suspension. In some embodiments, filtering may include a first filtering using a first temperature of about 0 to about 5° C., or 0° C. to about 10° C., and a second temperature of about 20° C. to about 30° C., or 15° C. to about 35° C. For example, filtering may include processing about 1 to about 6 diavolumes at about 0 to about 5° C., and processing at least one diavolume (e.g. about 1 to about 3 or about 1-2 diavolumes) at about 20° C. to about 30° C.

After purifying and concentrating the nanoparticle suspension, the particles may be passed through one, two or more sterilizing and/or depth filters, for example, using ˜0.2 μm depth pre-filter.

In exemplary embodiment of preparing nanoparticles, an organic phase is formed composed of a mixture of a therapeutic agent, e.g., bortezomib, and polymer (homopolymer, and co-polymer). The organic phase may be mixed with an aqueous phase at approximately a 1:5 ratio (oil phase:aqueous phase) where the aqueous phase is composed of a surfactant and optionally dissolved solvent. A primary emulsion may then formed by the combination of the two phases under simple mixing or through the use of a rotor stator homogenizer. The primary emulsion is then formed into a fine emulsion through the use of e.g. high pressure homogenizer. Such fine emulsion may then quenched by, e.g. addition to deionized water under mixing. An exemplary quench:emulsion ratio may be about approximately 8.5:1. A solution of Tween (e.g., Tween 80) can then be added to the quench to achieve e.g. approximately 2% Tween overall, which may serves to dissolve free, unencapsulated drug. Formed nanoparticles may then be isolated through either centrifugation or ultrafiltration/diafiltration.

In an embodiment, the formation and recovery of disclosed therapeutic particles is performed so as to limit the reconversion of the boronate ester compound back to the acid. In some embodiments, it may be desirable to substantially avoid formation of ionized forms of boronate ester compounds.

The following embodiments may apply to any of the methods of preparing disclosed herein. In one embodiment, the process may further include removing water from the reaction mixture. Water may be removed using any conventional process, including, but not limited to, use of molecular sieves, azeotropic distillation, or use of chemical drying agents including phosphorous pentoxide and calcium hydride. The emulsion phase may include preparing a primary emulsion, and emulsifying the primary emulsion to form a fine emulsion. Emulsifying may include use of a rotor stator homogenizer, probe sonicator, or a high pressure homogenizer. The second phase may include about 1 to about 40 weight % solids. The organic solvent may be ethyl acetate, benzyl alcohol, or a combination thereof. In another embodiment, the organic solvent may be ethyl acetate, benzyl alcohol, ethanol, isopropyl alcohol, acetone, toluene, dichloromethane, or hexafluoroisopropanol, or a combination thereof. The aqueous solution may be water, optionally having one or more of sodium cholate, ethyl acetate, and benzyl alcohol. The drug solubilizer may be selected from the group consisting of Tween 80, Tween 20, polyvinyl pyrrolidone, cyclodextran, sodium dodecyl sulfate, and sodium cholate.

For example, a biodegradable polymeric material can be mixed with boronate esters for encapsulation in a water miscible or partially water miscible organic solvent. In one embodiment, the biodegradable polymer can be any of the biodegradable polymers disclosed herein, for example, poly(D,L-lactic acid), poly(D,L-glycolic acid), poly(ε-caprolactone), or a block PEG-PLA copolymer. The water miscible organic solvent can be but is not limited to: acetone, ethanol, methanol, or isopropyl alcohol. The partially water miscible organic solvent can be, but is not limited to: acetonitrile, tetrahydrofuran, ethyl acetate, isopropyl alcohol, isopropyl acetate, or dimethylformamide. The resulting polymer solution can then added to the aqueous solution of conjugated and unconjugated amphiphilic compound to yield particles by the rapid diffusion of the organic solvent into the water and evaporation of the organic solvent.

### Compositions and Methods of Treatment

The present disclosure also provides pharmaceutical compositions comprising particles as disclosed herein formulated together with one or more pharmaceutically acceptable carriers. Exemplary materials which can serve as pharmaceutically acceptable carriers include, but are not limited to, sugars such as lactose, glucose, and sucrose; starches such as com starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyllaurate; agar; detergents such as TWEEN™ 80; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. If filtration or other terminal sterilization methods are not feasible, the formulations can be manufactured under aseptic conditions.

One embodiment provides a method of treating a disease or disorder selected from cancer, solid tumor cancer (such as laryngeal tumors, brain tumors, and other tumors of the head and neck; colon, rectal and prostate tumors; breast and thoracic solid tumors; ovarian and uterine tumors; tumors of the esophagus, stomach, pancreas and liver; bladder and gall bladder tumors; skin tumors such as melanomas; and the like), multiple myeloma, mantle cell lymphoma, and hematologic malignancy comprising administering to a patient in need thereof a composition disclosed herein. For example, provided herein is a method of treating multiple myeloma comprising administering to a patient in need thereof a composition of the invention. Moreover, a tumor or cancer treated can be either primary or a secondary tumor resulting from metastasis of cancer cells elsewhere in the body to e.g. the chest.

The pharmaceutical compositions can be administered to a patient by any means known in the art including oral and parenteral routes, and/or systemically, e.g., by IV infusion or injection. In one embodiment, the disclosed particles may be administered by IV infusion. In one embodiment, disclosed particles may be locally administered, for example, brought into contact with the blood vessel wall or vascular tissue through a device.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid can be used in the preparation of injectables. In one embodiment, the inventive conjugate is suspended in a carrier fluid comprising 1% (w/v) sodium carboxymethyl cellulose and 0.1% (v/v) TWEEN™ 80. The injectable formulations can be sterilized, for example, by filtration through a bacteria-retaining filter, or by incorporating sterilizing agents in the form of sterile solid compositions which can be dissolved or dispersed in sterile water or other sterile injectable medium prior to use.

Therapeutic particles disclosed herein may be formulated in dosage unit form for ease of administration and uniformity of dosage. The expression “dosage unit form” as used herein refers to a physically discrete unit of particle appropriate for the patient to be treated. It will be understood, however, that the total daily usage of the compositions of the present invention will be decided by the attending physician within the scope of sound medical judgment. For any particle, the therapeutically effective dose can be estimated initially either in cell culture assays or in animal models, usually mice, rabbits, dogs, or pigs. The animal model can be also used to achieve a desirable concentration range and route of administration. Such information can then be used to determine useful doses and routes for administration in humans. Therapeutic efficacy and toxicity of particles can be determined by standard pharmaceutical procedures in cell cultures or experimental animals, e.g., ED₅₀ (the dose is therapeutically effective in 50% of the population) and LD₅₀ (the dose is lethal to 50% of the population). The dose ratio of toxic to therapeutic effects is the therapeutic index, and it can be expressed as the ratio, LD₅₀/ED₅₀. Pharmaceutical compositions which exhibit large therapeutic indices may be useful in some embodiments. The data obtained from cell culture assays and animal studies can be used in formulating a range of dosage for human use.

The examples which follow are intended in no way to limit the scope of this invention but are provided to illustrate how to prepare and use compounds of the present invention. Many other embodiments of this invention will be apparent to one skilled in the art.

### EXAMPLES

The invention now being generally described, it will be more readily understood by reference to the following examples which are included merely for purposes of illustration of certain aspects and embodiments of the present invention, and are not intended to limit the invention in any way.

### Examples 1

A boronate ester compound is formed using a monoglyceride as shown in Scheme 1 and bortezomib. 1-undeconyl-rac-glycerol is reacted in an organic solution with bortezomib to yield a lipid-bortezomib suitable for encapsulation in a polymeric nanoparticle.

### Example 2

A boronate ester compound is formed using PEG-poly(depsipeptide) bearing a diol functionality as shown in Scheme 2 as a conjugating polymer. Morpholine-2,5-dione A (where R is CH₂CH₂COOBzl) and dioxane-2,5-dione B are reacted with HO-PEG-OMe to afford PEG-poly(depsipeptide) C, where x is 1 to about 1000 or to about 10,000. Acid activation of the terminal carboxylic acid groups (for example, using EDC-NHS conditions described above or any other conventional acid activation methods) followed by treatment with 3-amino-1,2-propanediol forms the amido diol for boronate ester formation D. Such a polymer may ultimately degrade into lactic acid, glycolic acid and amino acids (such as glutamic acid or aspartic acid) or its diol derivative.

Reaction of the boronic acid with the conjugating polymer can occur under similar conditions as that for low molecular weight diols. The drug-polymer conjugates can then be combined with a biodegradable polymer solution, such as PLGA-PEG copolymer at a concentration of about 20-30 wt. % or higher and nano-emulsion of the mixture encapsulates the conjugages to form nanoparticles. A concentration higher than the polymer overlap concentration may result in polymer chain entanglement, which limits the amount of polymer lost to the aqueous phase during nano-emulsion

### Example 3

Conjugation with bortezomib and a polymer bearing structural similarities to poly(hydroxypropylmethacrylamide) (HPMA) (where xis 1 to about 100 and y is 1 to about 100) is depicted in Scheme 3. This type polymer has been used as a conjugate to a number of known drugs, for example, HPMA-doxorubicin, HPMA-doxorubicin-galatosamine, HPMA-paclitaxel, HPMA-camptothesin, HPMA-cisplatinate by CRC/Pharmacea. Such polymer may degrade in vivo into amino acids and poly(2,3-dihydroxypropyl methacrylamide.

### Example 4

A boronate ester compound formed by radical copolymerization (where x is 1 to about 100 and y is 1 to about 100) is depicted in Scheme 4. After conjugation with bortezomib, for example, the polymer would be expected to degrade hydrolytically in vivo into poly(methacrylic acid), glycerol and butyl alcohol, for example.

### Example 5: Synthesis of PEG-Lipid-Bortezomib Conjugates

PEGylated lipids incorporate a diol functionality for boronate ester formation and a reactive moiety for poly(ethylene glycol) attachment. Suitable lipids for forming PEG-lipid-bortezomib conjugates include, for example, 10,11-dihydroxyundecanoic acid, 9,10,16-trihydroxyhexadecanoic acid (R, R; R, S; S, S). Poly(ethylene glycol) (Number Average Molar Mass=1 kDa, 2 kDa, 3.5 kDa, 5 kDa) bearing an amino-terminus can be used for synthesizing a PEGylated lipid which is then be converted to the boronate ester of bortezomib as depicted in Scheme 5.

A mixture of acids A and B are treated with one or more agents known in the art to protect the diol functionality. The acids are then activated and treated with a mixture of H₂N—(CH₂CH₂O)ₙ—OMe, where n is 23, 45, 80, and 114 to form amides C and D. The diol functional group of C and D is then deprotected using standard methods. Complexation of the free diol with bortezomib provides boronate esters E and F.

### Example 6: PEGylated-Lipid-Bortezomib Conjugate and Polymer Hybrid Nanoparticles

To form the nanoparticles, an organic solution containing the PEG-lipid-bortezomib conjugate and a homopolymer poly(ester) (PLGA, PLA, etc) or diblock copolymer based on PEG and poly(ester) (PLA-PEG, PLGA-PEG, etc) is emulsified into an aqueous phase. The emulsion is prepared by known methods, such as high pressure homogenization, and process parameters are optimized to yield nanoparticles in the 50-100 nm range.

The PEG-lipid-bortezomib conjugate may stabilize the oil-water interface in the emulsion due to the hydrophilic nature of PEG and its preference for the aqueous continuous phase. The organic solvent from the emulsion is extracted by quenching into a large volume of water leading to nanoparticle formation. Exemplary methods such as diafiltration are employed to remove water miscible solvent, yielding a solvent-free aqueous suspension of nanoparticles.

### Example 7A

Boronate esters of bortezomib and the backbone or side chain hydroxyl moieties of dextran are prepared in an organic solvent such as methyl sulfie, formamide or methylformamide, as depicted in Scheme 6:

A mixture of dextran-bortezomib conjugate solution and diblock copolymers (PEG-Poly(lactide) or PEG-Poly(lactide-co-glycolide)) solutions in partially water miscible organic solvents such as ethyl acetate or its binary mixtures with DMS are emulsified into an aqueous phase using known techniques such as high pressure homogenization. The removal of organic solvent by quenching into a large volume of water or by evaporation under reduced pressure yield nanoparticles based on a polyester core and polyethylene glycol corona. The dextran-bortezomib conjugate is expected to be trapped within the polymeric core due to slow diffusion properties of the macromolecular dextran-bortezomib conjugate. Such kinetic entrapment may occur despite a thermodynamic preference of the dextran-bortezomib conjugate for the water phase thereby improving the efficiency of encapsulation of bortezomib into the nanoparticles. Upon intravenous administration to a patient, the hydration of the nanoparticle core and resulting hydrolysis of the boronate ester may cause release of the bortezomib from the nanoparticle.

### Example 7B: Dextran-Graft-Polylactide Bortezomib Nanoparticles

Poly(lactide) (PLA) is first grafted onto a dextran backbone and then subsequently the vicinal hydroxyl groups on dextran are utilized for botezomib conjugation in a manner analogous to that described above. This may enhance the hydrophobic character of the dextran-bortezomib conjugates. Dextran-graft-PLA will be prepared, for example, using the process shown in scheme 7.

Dextran-graft-PLA are then utilized to prepare dextran-graft-PLA-Bortezomib conjugates in a manner analogous to that described above for dextran-bortezomib conjugates and then incorporated into nanoparticles by known emulsion methods.

### Example 8A: (Bortezomib)₂-pamoic Acid Ester

A 25 mL flask was charged with 0.85 grams of 4-[(3-Carboxy-2-hydroxynaphthalen-1-yl)methyl]-3-hydroxynaphthalene-2-carboxylic acid (pamoic acid) and 1.7 grams of Bortezomib. Ten grams of dimethylformamide was added and the mixture was stirred at room temperature. The reaction mixture clarified over time, and was complete in 2 hours by TLC. The reaction mixture was filtered using a 0.45 μm PVDF syringe filter and added dropwise to 250 mL ethyl ether under vigorous stirring resulting in the formation of a white precipitate. The solid was collected by vacuum filtration, transferred to a tared vial, and dried extensively under high vacuum to afford 2 grams of pure compound. TLC: 70:30 Acetone: Heptane Rf 0.5 on silica gel 60 F₂₅₄ plates

### Example 8B: Bortezomib-xinafoic Acid Ester

A 250 mL flask was charged with 2 grams of Bortezomib and 980 mg of 1-hydroxy-2-napthoic acid (xinafoic acid). 50 grams of ethyl acetate was added and the mixture was stirred at room temperature. The solution clarified over time, and was complete by TLC in 3 hours. The reaction solution was added to 500 mL of cold stirring heptane dropwise to afford a white precipitate. The solid was collected by vacuum filtration and dried extensively under high vacuum to afford 2.4 grams of pure compound. TLC: 70:30 Acetone: Heptane Rf 0.5 on silica gel 60 F₂₅₄ plates

### Example 9: PLA-Mannose-Bortezomib Conjugate Nanoparticles Based on a Poly(Ester Core) and a Poly(Ethylene Glycol) Corona

A PLA-mannose-bortezomib conjugate is prepared as in scheme 8:

PLA-sugar-bortezomib conjugates are then incorporated into nanoparticles based on PLA-PEG diblock copolymers by emulsification of a solution containing this polymer-drug conjugate and PLA-PEG diblock copolymers in partially water miscible organic solvent such as ethyl acetate into an aqueous phase and subsequent removal of organic solvent. The molecular weight of the PLA is optimized to the minimum chain length necessary to impart hydrophobic character to the conjugate while maximizing the weight fraction of drug in the polymer-drug conjugate to maximize final drug load in the nanoparticles.

### Example 10

PEGylation of dextran is conducted by a) backbone oxidation followed by reductive amination using amino-terminated PEG (top reaction scheme) and b) carbodimidazole activation and amide bond formation using amino-terminated PEG, as shown in Scheme 9:

PEG-dextran-bortezomib conjugates are prepared in a manner similar to that of the dextran-bortezomib conjugates described earlier. Nanoparticles based on the PEG-dextran-bortezomib conjugate may be prepared by an emulsion process using a partially water miscible organic solvent such as ethyl acetate in a manner analogous to that described above. Alternatively, the polymer drug conjugate may be prepared in a completely water miscible organic solvent such as dimethylsulfoxide, formamide, dimethylformamide, N-methylpyrrolidone, acetone, acetonitrile or their mixtures and subsequently added to an aqueous phase to obtain PEG stabilized colloidal nanoparticles or partially hydrated macromolecules depending upon the water solubility of the dextran-bortezomib conjugate. The resulting colloid may be comprised of a collapsed dextran-bortezomib segment and a soluble PEG segment of individual macromolecules in the sub-10 nm range. The soluble PEG segment is expected to sterically stabilize such polymer-drug conjugates and prevent aggregation/precipitation. Alternatively, assembly of several individual macromolecules may yield colloidal nanoparticles bearing a dextran-bortezomic core and a PEG corona. In either system, the PEG corona is expected to both stabilize the nano-carriers and prevent detection and elimination by the immune system upon intravenous delivery. Furthermore, such nanoparticles/polymer drug conjugates are expected to accumulate in solid tumors as a result of enhanced permeability and retention effect.

### Example 11: Bortezomib Nanoparticle Preparation

An organic phase is formed composed of a mixture of bortezomib (i.e. a non-esterified peptide boronic compound) and polymer (homopolymer, co-polymer, and optionally a co-polymer with ligand). An organic phase is mixed with an aqueous phase at approximately a 1:5 ratio (oil phase:aqueous phase) where the aqueous phase is composed of a surfactant and some dissolved solvent. In order to achieve high drug loading, about 30% solids in the organic phase is used.

First a primary, coarse emulsion is formed by the combination of the two phases under simple mixing or through the use of a rotor stator homogenizer. The rotor/stator yields a homogeneous milky solution, while the stir bar produces a visibly larger coarse emulsion. It is observed that the stir bar method resulted in significant oil phase droplets adhering to the side of the feed vessel, suggesting that while the coarse emulsion size is not a process parameter critical to quality, it should be made suitably fine in order to prevent yield loss or phase separation. Therefore the rotor stator is used as the standard method of coarse emulsion formation, although a high speed mixer may be suitable at a larger scale.

The primary emulsion is then formed into a fine emulsion through the use of a high pressure homogenizer. The size of the coarse emulsion does not significantly affect the particle size after successive passes (1-3) through the homogenizer.

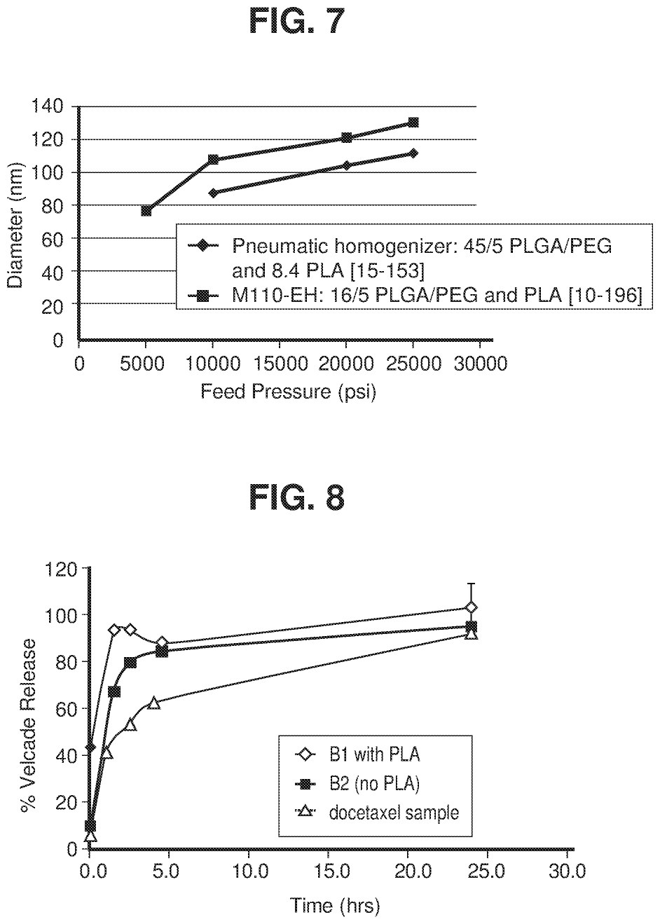

Homogenizer feed pressure is found to have a significant impact on resultant particle size. On both the pneumatic and electric M-110EH homogenizers, it is found that reducing the feed pressure also reduced the particle size (FIG. 7). Therefore the standard operating pressure used for the M-110EH is 4000-5000 psi per interaction chamber, which is the minimum processing pressure on the unit. The M-110EH also has the option of one or two interaction chambers. It comes standard with a restrictive Y-chamber, in series with a less restrictive 200 μm Z-chamber. It is found that the particle size was actually reduced when the Y-chamber was removed and replaced with a blank chamber. Furthermore, removing the Y-chamber significantly increases the flow rate of emulsion during processing.

After 2-3 passes the particle size is not significantly reduced, and successive passes can even cause a particle size increase.

The effect of scale on particle size shows surprising scale dependence. The trend shows that in the 2-10 g batch size range, larger batches produce smaller particles. It has been demonstrated that this scale dependence is eliminated when considering greater than 10 g scale batches. The amount of solids used in the oil phase is about 30% solids concentration on particle size. Table A summarizes the emulsification process parameters.

TABLE A

| Parameter | Value | Observation |
|---|---|---|
| Coarse emulsion | Rotor stator | Coarse emulsion size does not |
| formation | homogenizer | affect final particle size, but |
| | | large coarse emulsion can cause |
| | | increased oil phase retention in |
| | | feed vessel |
| Homogenizer | 4000-5000 psi | Lower pressure reduces particle |
| feed pressure | per chamber | size |
| Interaction | 2 × 200 μm | 200 μm Z-chamber yields the |
| chamber(s) | Z-chamber | smallest particle size, and allows |
| | | for highest homogenizer throughput |
| Number of | 2-3 passes | Studies have shown that the |
| homogenizer | | particle size is not significantly |
| passes | | reduced after 2 discreet passes, |
| | | and size can even increase with |
| | | successive passes |
| Water phase | 0.1% | [Sodium cholate] can effectively |
| [sodium cholate] | | alter particle size; value is |
| | | optimized for given process and |
| | | formulation |
| W:O ratio | 5:1 | Lowest ratio without significant |
| | | particle size increase is ~5:1 |
| [Solids] in oil | 30% | Increased process efficiency, |
| phase | | increased drug encapsulation, |
| | | workable viscosity |

The fine emulsion is then quenched by addition to deionized water at a given temperature under mixing. In the quench unit operation, the emulsion is added to a cold aqueous quench under agitation. This serves to extract a significant portion of the oil phase solvents, effectively hardening the nanoparticles for downstream filtration. Chilling the quench significantly improved drug encapsulation. The quench:emulsion ratio is approximately 5:1.

A solution of 35% (wt %) of Tween 80 is added to the quench to achieve approximately 2% Tween 80 overall After the emulsion is quenched a solution of Tween-80 is added which acts as a drug solubilizer, allowing for effective removal of unencapsulated drug during filtration. Table B indicates each of the quench process parameters.

TABLE B
Summary quench process parameters.

| Parameter | Value | Observation |
|---|---|---|
| Initial quench | <5° C. | Low temperature yields higher drug |
| temperature | | encapsulation |
| Tween-80 | 35% | Concentration that can be prepared |
| solution | | and readily disperses in quench |
| Tween-80:drug | 25:1 | Minimum amount of Tween-80 required |
| ratio | | to effectively remove unencapsulated |
| | | drug |
| Q:E ratio | 5:1 | Minimum Q:E ratio while retaining |
| | | high drug encapsulation |
| Quench | ≤5° C. (with | Temperature which prevents |
| hold/processing | current 5:1 | significant drug leaching during |
| temp | Q:E ratio, 25:1 | quench hold time and initial |
| | Tween- | concentration step |
| | 80:drug ratio) | |

The temperature must remain cold enough with a dilute enough suspension (low enough concentration of solvents) to remain below the T_{g} of the particles. If the Q:E ratio is not high enough, then the higher concentration of solvent plasticizes the particles and allows for drug leakage. Conversely, colder temperatures allow for high drug encapsulation at low Q:E ratios (to ˜3:1), making it possible to run the process more efficiently.

The nanoparticles are then isolated through a tangential flow filtration process to concentrate the nanoparticle suspension and buffer exchange the solvents, free drug, and drug solubilizer from the quench solution into water. A regenerated cellulose membrane is used with a molecular weight cutoff (MWCO) of 300.

A constant volume diafiltration (DF) is performed to remove the quench solvents, free drug and Tween-80. To perform a constant-volume DF, buffer is added to the retentate vessel at the same rate the filtrate is removed. The process parameters for the TFF operations are summarized in Table C. Crossflow rate refers to the rate of the solution flow through the feed channels and across the membrane. This flow provides the force to sweep away molecules that can foul the membrane and restrict filtrate flow. The transmembrane pressure is the force that drives the permeable molecules through the membrane.

TABLE C
TFF Parameters

| | Optimized | | |
|---|---|---|---|
| Parameter | Value | | Effect |
| Membrane | Regenerated | | No difference in performance |
| Material | cellulose - | | between RC and PES, but solvent |
| | Coarse | | compatibility is superior for RC. |
| | Screen | | |
| | Membrane | | |
| Molecular | 300 | kDa | No difference in NP |
| Weight Cut off | | | characteristics (i.e. residual |
| | | | tween)Increase in flux rates is |
| | | | seen with 500 kDa membrane but |
| | | | 500 kDa is not available in RC |
| Crossflow Rate | 11 | L/min/m² | Higher crossflow rate led to |
| | | | higher flux |
| Transmembrane | 20 | psid | Open channel membranes have |
| Pressure | | | maximum flux rates between 10 |
| | | | and 30 psid. Coarse channel |
| | | | membranes have maximum flux |
| | | | rates with min TMP (~20 psid). |
| Concentration | 30 | mg/ml | Diafiltration is most efficient |
| of Nanoparticle | | | at [NP] ~50 mg/ml with open |
| Suspension for | | | channel TFF membranes based on |
| Diafiltration | | | flux rates and throughput. With |
| | | | coarse channel membranes the |
| | | | flux rate is optimized at ~30 |
| | | | mg/ml in the starting buffer. |
| Number of | ≥15 (based | | About 15 diavolumes are needed |
| Diavolumes | on flux | | to effectively remove tween-80. |
| | increase) | | End point of diafiltration is |
| | | | determined by in-process control |
| | | | (flux increase plateau). |
| Membrane Area | ~1 | m²/kg | Membranes sized based on |
| | | | anticipated flux rates and volumes |
| | | | required. |

The filtered nanoparticle slurry is then thermal cycled to an elevated temperature during workup. A small portion (typically 5-10%) of the encapsulated drug is released from the nanoparticles very quickly after its first exposure to 25° C. Because of this phenomenon, batches that are held cold during the entire workup are susceptible to free drug or drug crystals forming during delivery or any portion of unfrozen storage. By exposing the nanoparticle slurry to elevated temperature during workup, this ‘loosely encapsulated’ drug can be removed and improve the product stability at the expense of a small drop in drug loading. 5 diavolumes is used as the amount for cold processing prior to the 25° C. treatment.

After the filtration process the nanoparticle suspension is passed through a sterilizing grade filter (0.2 μm absolute). Pre-filters are used to protect the sterilizing grade filter in order to use a reasonable filtration area/time for the process. Values are as summarized in Table D.

TABLE D

| Parameter | O Value | | Effect |
|---|---|---|---|
| Nanoparticle | 50 | mg/ml | Yield losses are higher at higher |
| Suspension | | | [NP], but the ability to filter at |
| Concentration | | | 50 mg/ml obviates the need to |
| | | | aseptically concentrate after |
| | | | filtration |
| Filtration flow | ~1.3 | L/min/m² | Filterability decreases as flow |
| rate | | | rate increases |

The filtration train is Ertel Alsop Micromedia XL depth filter M953P membrane (0.2 μm Nominal); Pall SUPRAcap with Seitz EKSP depth filter media (0.1-0.3 μm Nominal); Pall Life Sciences Supor EKV 0.65/0.2 micron sterilizing grade PES filter.

0.2 m² of filtration surface area per kg of nanoparticles for depth filters and 1.3 m² of filtration surface area per kg of nanoparticles for the sterilizing grade filters can be used.

### Example 12: Nanoparticles with Bortezomib

Nanoparticles are prepared using the method of Example 11:

| | | Composition by Wt. |
|---|---|---|
| Lot No. | Components | (%) |
| 2 | mPEG(5k)-/PLA(16K)/Bortezomib | 97/3 |
| 3 | mPEG(5k)-/PLA(16K)/Bortezomib | 97/3 |
| 1 | mPEG(5k)-/PLA(16K)/Bortezomib | 96/4 |

| | | | Encapsulation |
|---|---|---|---|
| Lot No. | Size (nm) | Drug Load (%) | Efficiency (%) |
| 2 | 84 | 3.0 | 15.2 |
| 3 | 77 | 3.3 | 16.3 |
| 1 | 92 | 3.7 | 18.7 |

### Analytical Characterization

### Example 13: In Vitro Release

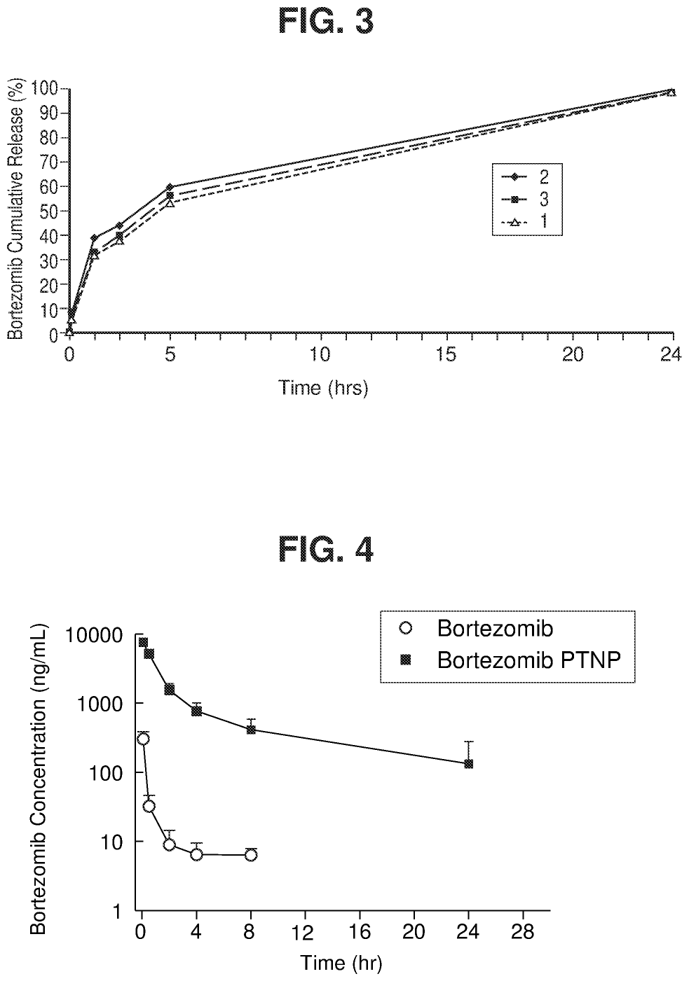

An in vitro release method is used to determine the initial burst phase release from nanoparticles at both ambient and 37° C. conditions. In order to maintain sink conditions and prevent nanoparticles from entering the release samples, a dialysis system is designed. After obtaining an ultracentrifuge capable of pelleting 100 nm particles, the dialysis membranes are eliminated and centrifugation is used to separate released drug from encapsulated drug. The dialysis system is as follows: 3 mL slurry of bortezomib nanoparticles (approx 250 μg/mL bortezomib PLGA/PLA nanoparticles, corresponding to 2.5 mg/mL solid concentration) in DI-water is placed into the inner tube of a 300 kDa MWCO dialyzer by pipetting. The nanoparticle is suspension in this media. The dialyzer is placed into a glass bottles containing 130 ml release media (2.5% hydroxyl beta cyclodextrin in PBS), which is continually stirred at 150 rpm using a shaker to prevent the formation of an unstirred water layer at the membrane/outer solution interface. At pre-determined time points, aliquot of samples (1 mL) were withdrawn from the outer solution (dialysate) and analyzed for bortezomib concentration by HPLC. FIG. 3 shows the in vitro release of the formulation of Example 12.

### Example 14: Pharmacologic Kinetic Profile

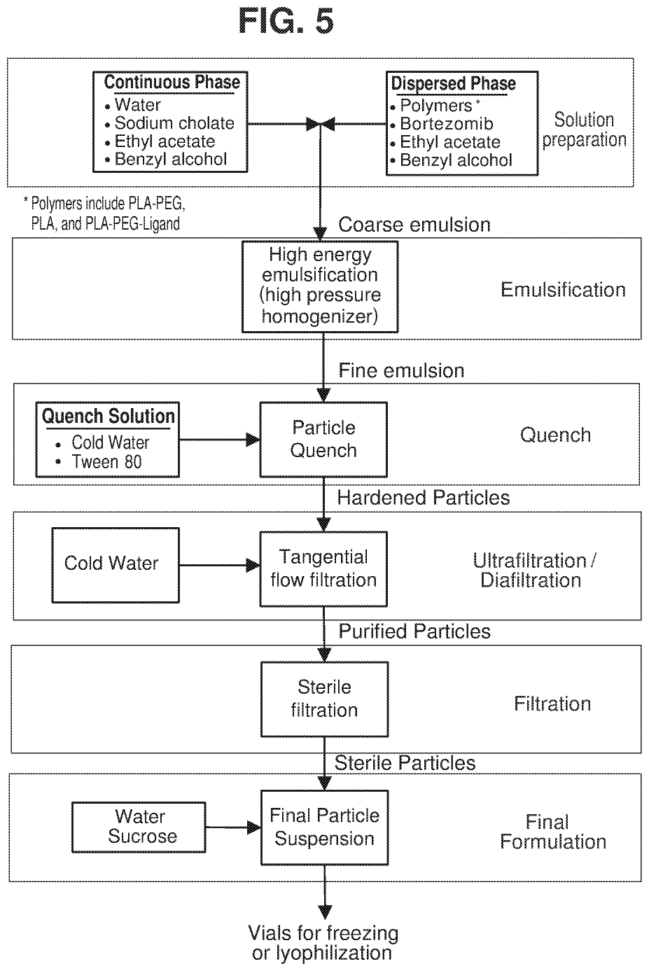

FIG. 4 depicts the pharmacologic kinetic profile of a single dose (0.5 mg/kg) of a formulation of Example 12 in Sprague-Dawley rats, compared to the profile of bortezomib alone.

### Example 15: Bortezomib Solubility

Solubility of bortezomib in ethyl acetate (EA), benzyl alcohol (BA), water, and 0.5% sodium cholate, 2% BA, 4% EA in water is measured. Results as shown in Table E demonstrate that bortezomib is soluble in these solvents.

TABLE E

| | Oil phase: | | Water phase: |
|---|---|---|---|
| | Benzyl alcohol:Ethyl | Quench | 0.5% sodium |
| | acetate | phase: | cholate, 2% BA, |
| | (BA:EA = 21:79, w/w %) | water | 4% EA in water |
| BTZ | 93 mg/ml < BTZ > | 1.1 mg/ml | 1.3 mg/ml |
| solubility | 112 mg/ml | | |

### Example 16: Bortezomib Formulations

Bortezomib formulations are prepared using PLA-PEG copolymer with and without PLA homopolymer (molecular weight 10,000 Mn). Briefly, batches are produced using a solvent system comprising 21% benzyl alcohol and 79% ethyl acetate (w/w). About 30% total solids in the oil phase is used. Analytical characterization of the bortezomib formulations with and without PLA is shown in Table F.

TABLE F

| | Formulation | | | | | |
|---|---|---|---|---|---|---|
| | PEG | Polymer | Target | Solid | Size | Loading |
| Number | Polymer | additives | API % | Conc | (nm) | (%) |
| B1 | 16/5 | 10 kDa | | 30% | 107 | 3.0 |
| | | PLA | | | | |
| | 40 | 40 | 20 | | | |
| B2 | 16/5 | no PLA | | 30% | 84 | 3.0 |
| | 80 | | 20 | | | |

The addition of PLA homopolymer increases particle size from 84 nm to about 107 nm but has little effect on bortezomib loading at the 30% oil phase solids concentration.

In vitro release test is performed on the B1 and B2 lots to determine the rate of drug release from the nanoparticles. Due to the high solubility of bortezomib in water, no solubilizer is included in the release medium to maintain sink condition. Thus PBS is used as the release medium.

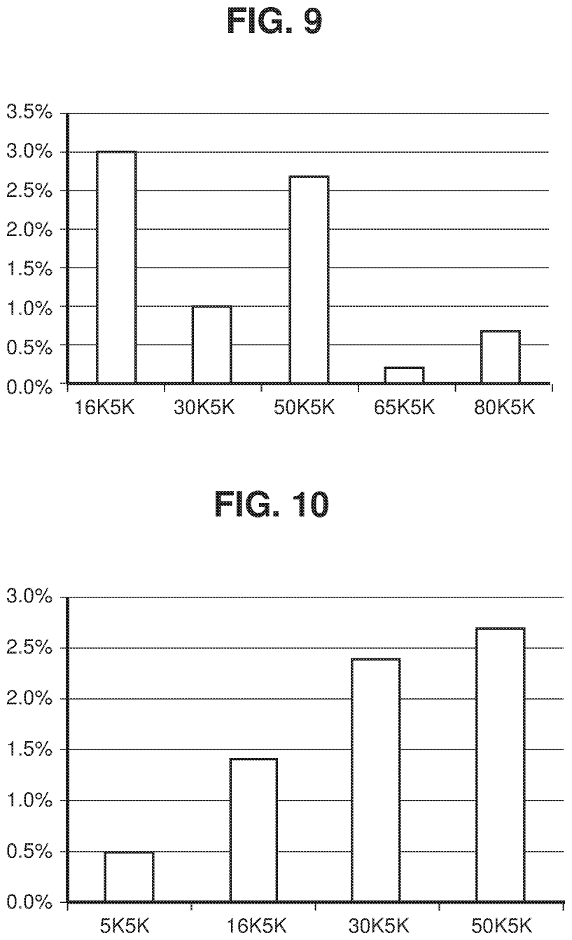

FIG. 8 depicts the in vitro release profiles of these nanoparticles. The bortezomib formulation prepared using PLA-PEG copolymer and PLA homopolymer appears to be a faster releasing formulation than the formulation without the PLA homopolymer. Thus, the addition of low molecular weight PLA homopolymer seems to increase the release rate of bortezomib. Further, the release of bortezomib from nanoparticles with PLA homopolymer is over 40% at time zero, indicating that bortezomib is loosely bound to the surface of nanoparticles. Finally, the two bortezomib nanoparticles show much faster drug release when compared to docetaxel nanoparticles.

### Example 17: Bortezomib Nanoparticles

Bortezomib nanoparticles are prepared using:

(1) PLA/PEG copolymers with molecular weights ranging from 5K/5K to 80K/5K PLA/PEG;

(2) incorporation of high molecular weight PLA homopolymer (80 kDa) with various PLA/PEG copolymers;

(3) incorporation of PLA homopolymer with amine end group (4K amine PLA) with the high molecular weight 65K/5K PLA/PEG copolymer;

(4) polycaprolactone PCL/PEG as copolymer (16K/5K PCL/PEG); and

(5) poly(lactic-glycolic acid) PLGA/PEG as copolymer (28K/5K PLGA/PEG).

Homopolymers are incorporated into these formulations at a 50:50 ratio with the PLA/PEG copolymer. Further, when either high molecular weight copolymer or homopolymer is used, the concentration of the sodium cholate surfactant in the water phase is increased to 5% (as compared to 0.5%) in order to obtain particle sizes of around 100 nm. Without changing surfactant concentration, the incorporation of high molecular weight PLA and PLA/PEG will result in larger particle size if all other variables are kept constant.

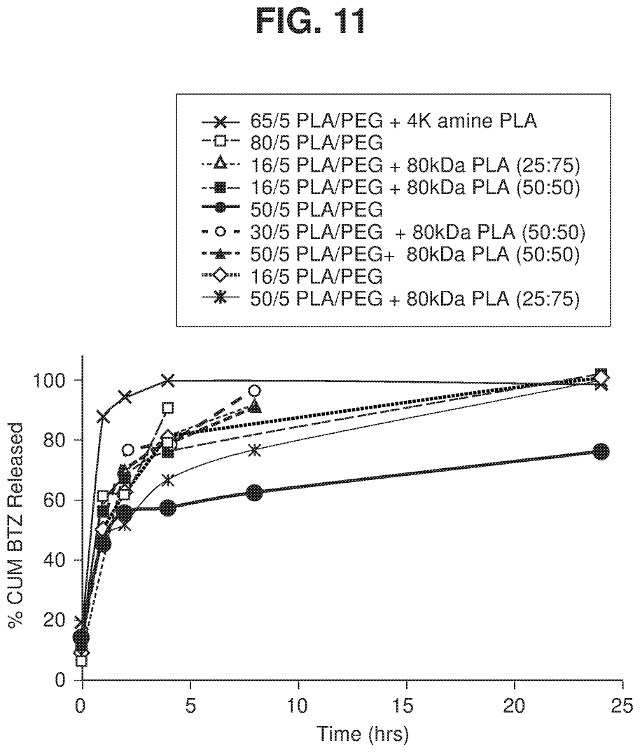

An evaluation of nanoparticles prepared using PLA/PEG copolymers with varying molecular weights show that inclusion of larger PLA blocks, which are more hydrophobic, does not result in a clear trend of higher drug loading. As depicted in FIG. 9, nanoparticles comprising 16/5 and 50/5 PLA/PEG show the highest drug load.

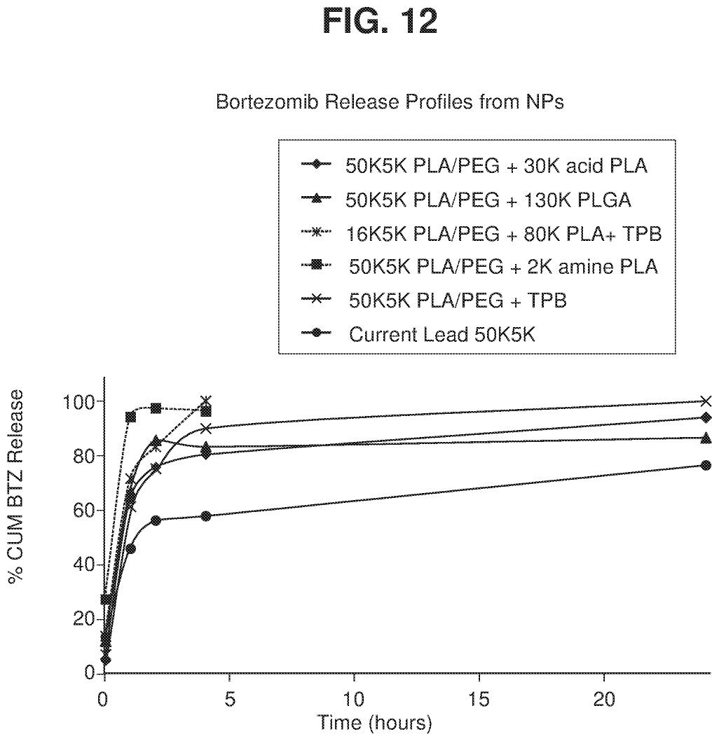

FIG. 10 depicts the drug loading of bortezomib formulations prepared using PLA/PEG copolymers of varying molecular weights and an 80 kDa PLA homopolymer.

Polycaprolactone PCL/PEG or poly(lactic-glycolic acid) PLGA/PEG as copolymers in nanoparticles are evaluated. The PCL/PEG copolymer contains polycaprolactone, which is more hydrophobic and not as soluble in ethyl acetate/benzyl alcohol as PLA. PCL is a common biodegradable polymer that has been used in FDA approved products. PLGA is a random copolymer of lactic and glycolic acid. Table G shows the results from bortezomib formulations incorporating either the PCL/PEG or the PLGA/PEG copolymers. Further, the incorporation of a PLA homopolymer with amine end group (4K amine PLA) with the high molecular weight 65K/5K PLA/PEG copolymer is also evaluated.

TABLE G

| | Copolymer | No | Homopolymer: | Homopolymer: | Homopolymer: |
|---|---|---|---|---|---|
| Copolymer | MW (Mn) | homopolymer | 10 kDa PLA | 80 kDa PLA | 4K amine PLA |
| PLA/PEG | 16K5K | 3% | 3% | 1.4% | ND |
| | 65K5K | 0.2% | ND | ND | 3.7% |
| PCL/PEG | 16K5K | 0.01% | ND | ND | ND |
| PLGA/PEG | 28K5K | 0.04% | ND | ND | ND |

Notably, the incorporation of the 4K amine PLA to 65K/5K PLA/PEG increased bortezomib loading significantly from 0.2% to 3.7%. It is believed that the amine-terminated PLA may ionically interact with the bortezomib and slow its release from nanoparticles.

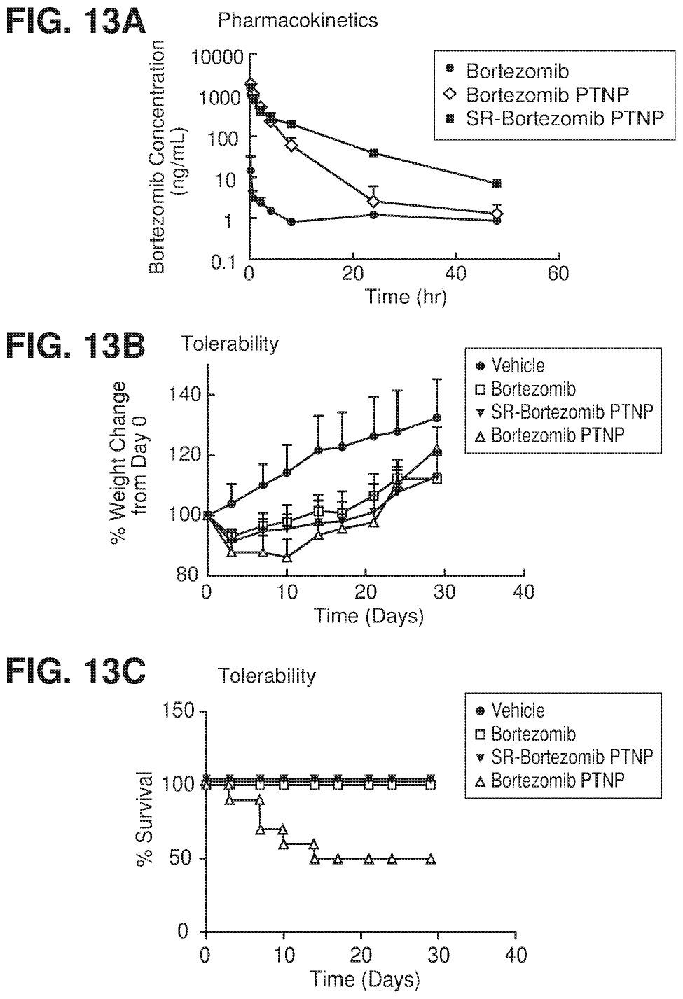

An in vitro release test is performed on bortezomib formulations with at least a 0.5% drug loading. PBS is again used as the release medium, as depicted in FIG. 11.

### Example 18: Bortezomib Nanoparticles

The following nanoparticle formulations are undertaken:

(1) incorporation of PLA homopolymer with amine end group (2K amine-PLA) with high molecular weight copolymer (50/5 PLA/PEG);

(2) incorporation of PLA homopolymer with carboxylic end group (30K COOH PLA) with high molecular weight copolymer (50K5K PLA/PEG);

(3) incorporation of PLGA copolymer with high molecular weight (130K PLGA) with high molecular weight copolymer (50/5 PLA/PEG);

(4) incorporation of sodium tetraphenylborate (TPB) with low and high molecular weight copolymers (16/5 and 50/5 PLA/PEG). It is believed that TPB may interact with bortezomib resulting in a decrease in drug solubility followed by slower drug release.

Homopolymers are incorporated into these formulations at a 50:50 ratio with the PLA/PEG copolymer. The 130K PLGA copolymer is also added at a 50:50 ratio with the PLA/PEG copolymer. Table H shows the results from bortezomib formulations using the above approaches.

TABLE H

| | | Doped | | TPB:Drug | BTZ | NP size |
|---|---|---|---|---|---|---|
| Formulation | Copolymer | polymer | | Molar ratio | loading (%) | (nm) |
| 1 | 50/5 PLA/PEG | — | | — | 2.70 | 106 |
| 2 | 50/5 PLA/PEG | 2K | amine PLA | — | 2.96 | 86.4 |
| 3 | 50/5 PLA/PEG | 30K | acidic PLA | — | 1.55 | 118.2 |
| 4 | 50/5 PLA/PEG | 130K | PLGA | — | 0.32 | 217.7 |
| 5 | 50/5 PLA/PEG | — | | 1:1 | 0.21 | 178.4 |
| 6 | 16/5 PLA/PEG | 80K | PLA | 1:1 | 0.18 | 206.5 |

Bortezomib formulations which incorporated the 2K amine PLA with 50/5 PLA/PEG show slightly higher drug load compared to the formulation with 50/5 PLA/PEG alone. In addition, the incorporation of the 130K PLGA copolymer increases the size of the nanoparticles from 106 nm to about 217.7 nm. This appears due to the fact that incorporation of high molecular weight polymers usually produces larger particle size if the concentration of surfactant is unchanged. Lastly, addition of TPB to the bortezomib formulation results in a decrease in drug load regardless of the types of PLA/PEG used.

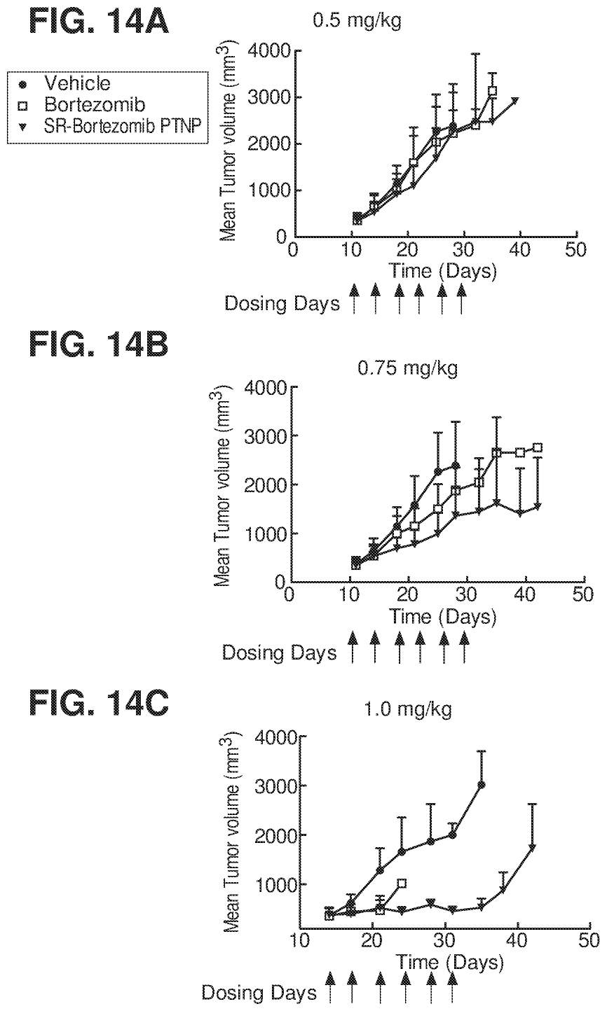

In vitro release test is performed on the above described bortezomib formulations, as depicted in FIG. 12.

### Example 19: Pharmacologic Kinetic and Tolerability Profiles

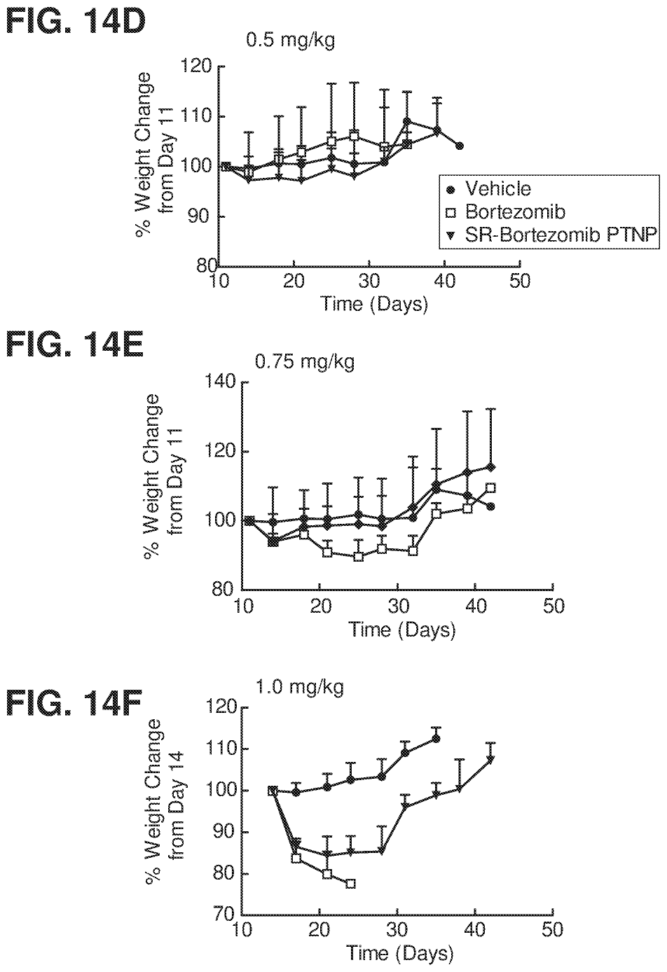

As shown in FIG. 13A, the pharmacologic kinetic profiles of nanoparticles containing bortezomib as prepared in Examples 12 and 17 are determined in Sprague-Dawley (SD) rats. Rats (male Sprague Dawley, approximately 300 g with jugular cannulae are given a single intravenous dose of 0.1 mg/kg free drug or passively targeted nanoparticles (PTNP) encapsulating drug at time t=0. At various times after dosing, blood samples are collected from the jugular cannulae into tubes containing lithium heparin, and plasma is prepared. Plasma levels are determined by extraction of the drug from plasma followed by LCMS analysis. The data shown in FIG. 13A indicates that the longest circulating particles are the SR-Bortezomib nanoparticles. However all formulations do successfully exhibit prolonged circulation times, as free drug is rapidly cleared from the plasma.

Drug tolerability is also determined in mice given an intravenous dose of 1.0 mg/kg free drug or passively targeted nanoparticles encapsulating drug twice a week for three weeks. Drug tolerability is assessed by weight change (FIG. 13B) and overall survival rate (FIG. 13C). As indicated in FIGS. 13B and 13C, the SR-Bortezomib nanoparticles appear to be well tolerated and have similar tolerability profiles as the free drug.

### Example 20: NCI-H460 Tumor Model

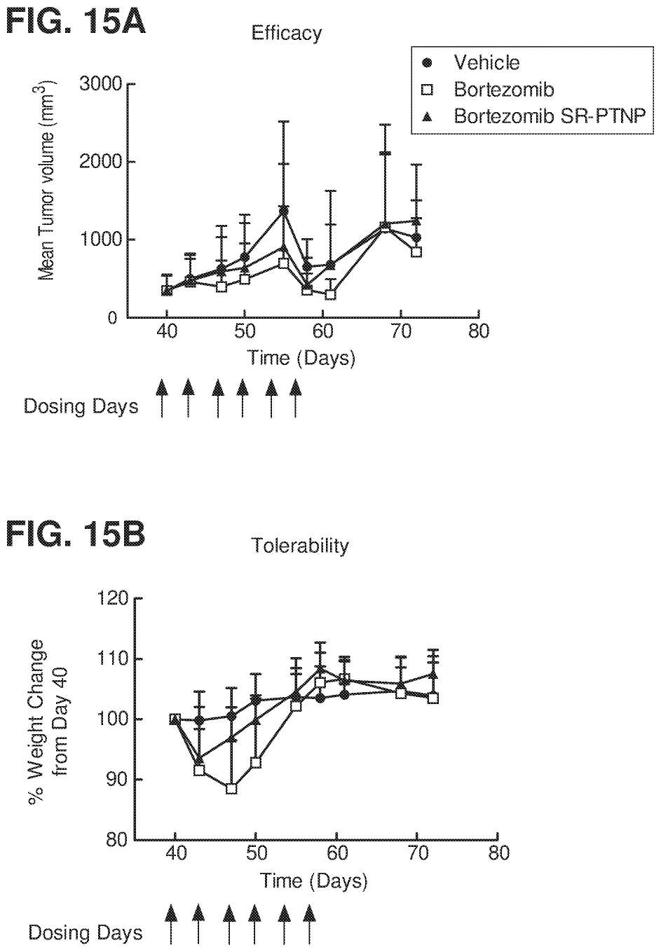

The ability of repeat doses of nanoparticles encapsulating bortezomib to suppress tumor growth is assessed in the NCI-H460 xenograft tumor model as shown in FIGS. 14A-C. Male Nu/Nu mice are subcutaneously inoculated with human NCI-H460 non-small cell lung cancer cells. Ten days after inoculation, the mice are treated twice weekly for three weeks with SR-bortezomib PTNP (as prepared in Example 17), free drug, or vehicle (Control). After six doses at either 0.5 mg/kg, 0.75 mg/kg, or 1.0 mg/kg, tumor volume is measured. Tumor volume reduction is greatest in animals receiving SR-bortezomib PTNP at all the dosages studied. As depicted in FIGS. 14D-F, treatment using SR-bortezomib PTNP also appears to be well tolerated compared to treatment using the free drug.

### Example 21: RPMI-8226 Tumor Model

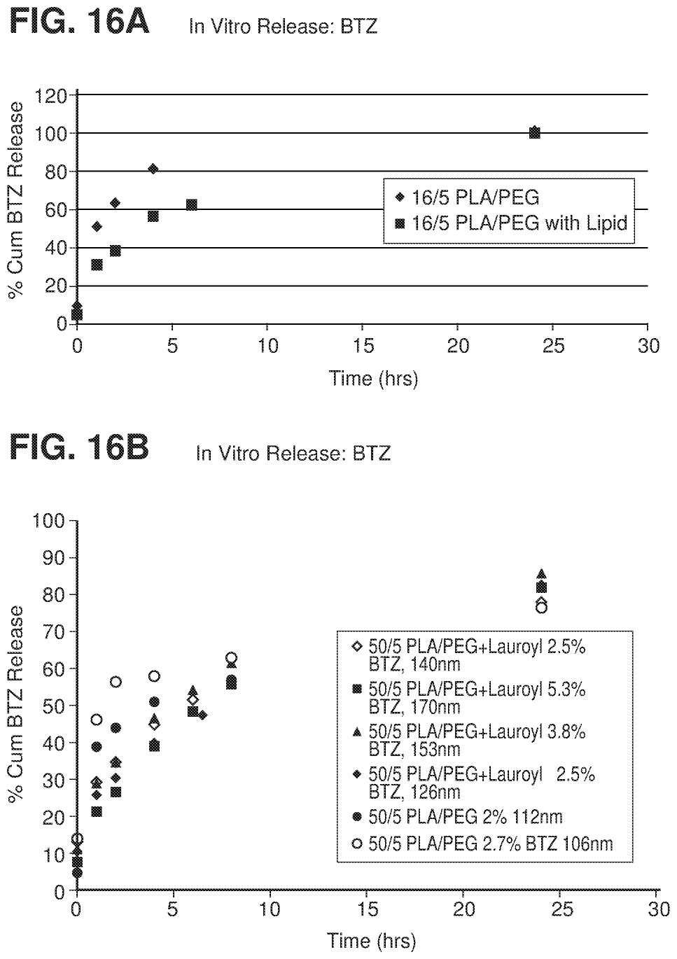

The ability of repeat doses of nanoparticles encapsulating bortezomib to suppress tumor growth is assessed in the RPMI-8226 xenograft tumor model as shown in FIG. 15A. Male Nu/Nu mice are subcutaneously inoculated with human RPMI-8226 multiple myeloma cells. Approximately forty days after inoculation, the mice are treated twice weekly for three weeks with SR-bortezomib PTNP (as prepared in Example 17), free drug, or vehicle (Control). After six doses at 1.0 mg/kg, tumor volume and drug tolerability are assessed. As depicted in FIGS. 15A and B, SR-bortezomib PTNP exhibits similar tumor reduction capability and drug tolerability profile as the free drug and vehicle control in this tumor model.

### Example 22: Bortezomib Nanoparticles

Bortezomib nanoparticles were prepared by blending PLA-PEG copolymer with monoglycerol lipids using the following formulation: 30% theoretical (w/w) drug; 70% (w/w) polymer, (PEG (16/5 PLA-PEG or 50/5 PLA-PEG) and lipid (monoglyceride). % Total solids=20%; solvents: 21% benzyl alcohol and 79% ethyl acetate (w/w).

For a 1 gram batch size, 300 mg of drug was mixed with 700 mg of a blend of Polymer-PEG (16-5 or 50-5 PLA-PEG) and lipid.

Bortezomib nanoparticles comprising monoglycerol lipids were produced as follows. In order to prepare a drug/polymer solution, appropriate amounts of bortezomib, polymer, and lipids were added to a 25 mL glass vial along with 3.16 g of ethyl acetate and 0.84 g of benzyl alcohol. The mixture was vortexed until the drug, polymer, and lipids were completely dissolved.

An aqueous solution for either a 16-5 PLA-PEG formulation or a 50-5 PLA-PEG formulation was prepared. The 16-5 PLA-PEG formulation contained 0.05% sodium cholate, 2% benzyl alcohol, and 4% ethyl acetate in water. Specifically, 0.5 g of sodium cholate and 939.5 g of DI water were added to a 1 L bottle and mixed using a stir plate until they were dissolved. Subsequently, 20 g of benzyl alcohol and 40 g of ethyl acetate were added to the sodium cholate/water mixture and mixed using a stir plate until all were dissolved. The 50-5 formulation contained 0.25% sodium cholate, 2% benzyl alcohol, and 4% ethyl acetate in water. Specifically, 2.5 g of sodium cholate and 937.5 g of DI water were added to a 1 L bottle and mixed using a stir plate until they were dissolved. Subsequently, 20 g of benzyl alcohol and 40 g of ethyl acetate were added to the sodium cholate/water mixture and mixed using a stir plate until all were dissolved.

An emulsion was formed by combining the organic phase into the aqueous solution at a ratio of 5:1 (aqueous phase:oil phase). The organic phase was poured into the aqueous solution and homogenized using hand homogenizer for 10 seconds at room temperature to form a coarse emulsion. The solution was subsequently fed through a high pressure homogenizer (110S). For the 16-5 PLA-PEG formulation, the pressure was set to 45 psi on gauge for two discreet passes to form the nanoemulsion. For the 50-5 PLA-PEG formulation, the pressure was set to 45 psi on gauge for two to four discreet passes to form the nanoemulsion.

The emulsion was quenched into cold DI water at <5° C. while stirring on a stir plate. The ratio of Quench to Emulsion was 8:1. 35% (w/w) Tween 80 in water was then added to the quenched emulsion at a ratio of 25:1 (Tween 80:drug).

The nanoparticles were concentrated through tangential flow filtration (TFF) followed by diafiltration to remove solvents, unencapsulated drug and solubilizer. A quenched emulsion was initially concentrated through TFF using a 300 KDa Pall cassette (2 membrane) to an approximately 100 mL volume. This was followed by diafiltration using approximately 20 diavolumes (2 L) of cold DI water. The volume was minimized by adding 100 mL of cold water to the vessel and pumping through the membrane for rinsing. Approximately 100-180 mL of material were collected in a glass vial. The nanoparticles were further concentrated using a smaller TFF to a final volume of approximately 10-20 mL.

In order to determine the solids concentration of unfiltered final slurry, a volume of final slurry was added to a tared 20 mL scintillation vial and dried under vacuum on lyo/oven. Subsequently the weight of nanoparticles was determined in the volume of the dried down slurry. Concentrated sucrose (0.666 g/g) was added to the final slurry sample to attain a final concentration of 10% sucrose.

In order to determine the solids concentration of 0.45 μm filtered final slurry, a portion of the final slurry sample was filtered before the addition of sucrose using a 0.45 μm syringe filter. A volume of the filtered sample was then added to a tared 20 mL scintillation vial and dried under vacuum on lyo/oven. The remaining sample of unfiltered final slurry were frozen with sucrose.

The following batches of bortezomib nanoparticles were produced, as shown in Table I.

TABLE I

| 35% Lipid + | | | | | |
|---|---|---|---|---|---|
| 35% 16/5 | | 35% Lipid + | | | |
| PLA/PEG + | | 35% 50/5 PLA/PEG + | | | |
| 30% BTZ | | 30% BTZ | | | |
| Lot # | | | | | |
| 82- | 82- | 82- | 82- | 82- | 82- |
| 130-2 | 170-6 | 170-1 | 170-2 | 170-3 | 180-2A |

| | | |
|---|---|---|
| BTZ | 30% (300 mg) | 30% (300 mg) |
| Polymer | 35% (350 mg) | 35% (350 mg) |
| PLA/PEG | 16/5 | 50/5 PLA/PEG |
| | PLA/PEG | |
| Lauroyl-rac | 35% (350 mg) | 35% (35 mg) |
| Glycerol | | |

Table J provides the particle size and drug load of the bortezomib nanoparticles described above.

TABLE J

| | 35% Lipid + | | | | | |
|---|---|---|---|---|---|---|
| | 35% 16/5 | | 35% Lipid + | | | |
| | PLA/PEG + | | 35% 50/5 PLA/PEG + | | | |
| | 30% BTZ | | 30% BTZ | | | |
| | Lot # | | | | | |
| | 82- | 82- | 82- | 82- | 82- | 82- |
| | 130-2 | 170-6 | 170-1 | 170-2 | 170-3 | 180-2A |
| Load | 18.06 | 12.12 | 2.46 | 5.34 | 3.83 | 2.46 |
| (%) | | | | | | |
| Size | 117.70 | 146.90 | 139.90 | 169.90 | 153.30 | 126.40 |
| (nm) | | | | | | |

Incorporation of 16-5 PLA-PEG and the monoglycerol lipid, lauroyl-rac-glycerol, into bortezomib nanoparticles appeared to result in higher drug encapsulation efficiency and higher drug loading. As shown in Table J, bortezomib nanoparticles comprising 16-5 PLA-PEG and lauroyl-rac-glycerol resulted in a drug load of more than 12%.

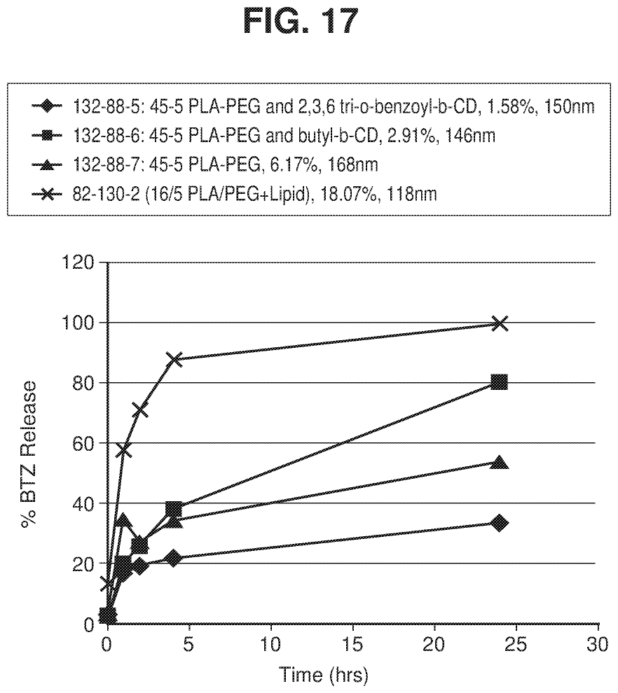

In vitro release test is performed on the above described bortezomib nanoparticles. As depicted in FIG. 16A and 16B, incorporation of either the 16-5 PLA-PEG or 50-5 PLA-PEG in combination with lauroyl-rac-glycerol slowed down the release of bortezomib from the nanoparticles compared with nanoparticles without lipids.

### Example 23 Bortezomib, PLA-PEG and β-CD Nanoemulsion Process (1 Gram Scale)

An organic solution, drug in solvent, is prepared as follows. To a 20 mL glass vial weight out and add 100 mg of BTZ (bortezomib). To the 20 mL glass vial weigh out and add 600 mg 16/5 PLA/PEG (PS). To the 20 mL glass vial weigh out and add 300 mg of β-CD. Add 4 grams of BA/EA mixture (21/79 wt ratio) and vortex until polymer is dissolved.

An aqueous solution is then prepared: 16-5 PLA-PEG formulation: 0.05% Sodium Cholate, 2% Benzyl Alcohol, 4% Ethyl acetate in Water. To 1 L bottle add 0.5 g sodium cholate and 939.5 g of DI water and mix on stir plate until dissolved. Add 20 g of benzyl alcohol and 40 g of ethyl acetate to sodium cholate/water and mix on stir plate until dissolved.

45-5 PLA-PEG formulation: 1% Sodium Cholate, 2% Benzyl Alcohol, 4% Ethyl acetate in Water: To 1 L bottle add 10 g sodium cholate and 930 g of DI water and mix on stir plate until dissolved. Add 20 g of benzyl alcohol and 40 g of ethyl acetate to sodium cholate/water and mix on stir plate until dissolved.

Formation of emulsion. Ratio of Aqueous phase to Oil phase is 5:1

Pour organic phase into aqueous solution and homogenize using hand homogenizer for 10 seconds at room temperature to form course emulsion. Feed solution through high pressure homogenizer (110S)

For a 16-5 PLA-PEG formulation set pressure to 45 psi on gauge for 1 or 2 discreet passes to form nanoemulsion (Target 120 nm for 16-5 and 150 nm for 45-5)

Formation of nanoparticles: Pour emulsion into Quench (D.I. water) at <3 C while stirring on stir plate. Ratio of Quench to Emulsion is 10:1. Add 35% (w/w) Tween 80 in water to quench at ratio of 25:1 Tween 80 to drug. Concentrate quench on TFF with 300 kDa Pall cassette (2 membrane) to ˜100 mL. Diafilter ˜20 diavolumes (2 liter) of cold DI water. Bring volume down to minimal volume. Add 100 mL of cold water to vessel and pump through membrane to rinse. Collect material in glass vial, 80-100 mL. Further concentrate the nanoparticle on a smaller TFF to a final volume of 10-20 mL

Determination of solids concentration of unfiltered final slurry: To tared 20 mL scintillation vial add a volume of final slurry and dry under vacuum on lyo/oven. Determine weight of nanoparticles in the volume of slurry dried down. Add concentrated sucrose (0.666 g/g) to final slurry sample to attain 10% sucrose. Determination of solids concentration of 0.45 um filtered final slurry: Filter about a portion of the final slurry sample before addition of sucrose through 0.45 μm syringe filter. To tared 20 mL scintillation vial add a volume of filtered sample and dry under vacuum on lyo/oven. Freeze remaining sample of unfiltered final slurry with sucrose.

Bortezomib with hydrophobic β-cyclodextrin formulation is prepared using 10% (w/w) theoretical drug, 30% (w/w) hydrophobic cyclodextrin: Heptakis(2,3,6-tri-O-benzoyl)-β-cyclodextrin, Triacetyl-β-cyclodextrin and Butyl-β-cyclodextrin, 60% (w/w) Polymer-PEG, (47-5 and 16-5 PLA-PEG), % Total Solids=20%; Solvents: 21% benzyl alcohol, 79% ethyl acetate (w/w).

Bortezomib formulations using hydrophobic B-CD were made using 45-5 PLA-PEG polymer and both 2,3,6 tri-o-benzoyl-β-CD and butyl-β-CD. The particle size and loading can be found in Table W. Note that the loading for the β-CD formulations is low because the theoretical drug load is lower than the 45-5 PLA-PEG formulation with no B-CD (10% compared to 30%). Overall, the particle size and processing was similar to the 45-5 PLA-PEG control.

TABLE W
Loading and size data of the 132-88 series
with Bortezomib and hydrophobic β-CDs

| | Drug | | | |
|---|---|---|---|---|
| | theoretical | Solid | | size |
| Lot # | loading | con | Loading % | (nm) |
| 132-88-5:45-5 PLA-PEG | 10 | 20% | 1.58 | 150 |
| and 2,3,6 tri-o-benzoyl-b- | | | | |
| CD, Bortezomib | | | | |
| 132-88-6:45-5 PLA-PEG | 10 | 20% | 2.91 | 146 |
| and butyl-b-CD, | | | | |
| Bortezomib | | | | |
| 132-88-7:45-5 PLA-PEG, | 30 | 20% | 6.17 | 168 |
| Bortezomib | | | | |

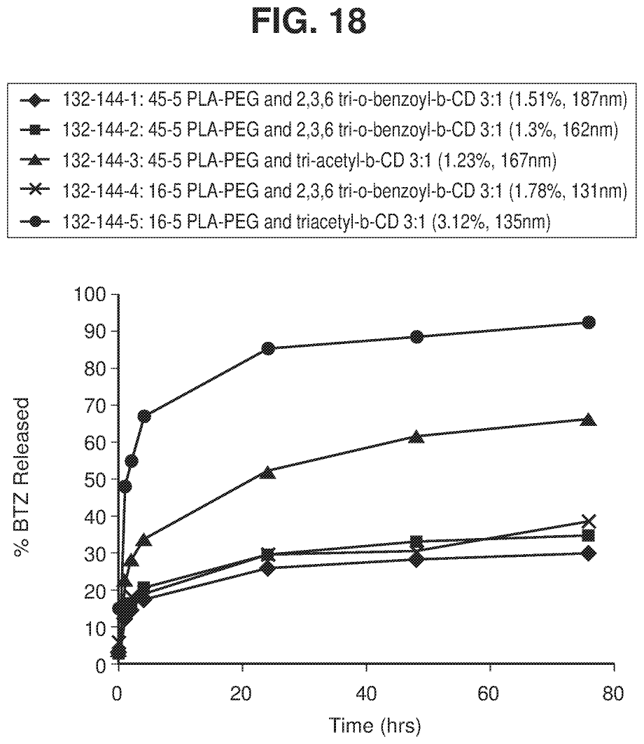

The in vitro release for these samples showed slower results (FIG. 17.) Similar to the Rofecoxib results presented below, the 45-5 PLA-PEG with 2,3,6 tri-o-benzoyl-β-CD showed the slowest in vitro release. Formulations using butyl-β-CD, which was chosen due to the presence of hydroxyl groups on the ring, showed faster release than the control 45-5 PLA-PEG formulation.

Other formulations were prepared and tested the encapsulation of Bortezomib with both 2,3,6 tri-o-benzoyl-β-CD and triacetyl-β-CD with 16-5 and 45-5 PLA-PEG polymers. These formulations showed similar loadings and sizes to the 132-88-series indicating that the runs can be reproducible. The results can be found in Table X.

TABLE X
Loading and size data of the 132-144 series
with Bortezomib and hydrophobic β-CDs

| | Drug | | | |
|---|---|---|---|---|
| | theoretical | Solid | | size |
| Lot # | loading | con | Loading % | (nm) |
| 132-144-1:BTZ+ 45-5 PLA- | 10 | 20% | 1.51 | 187 |
| PEG and 2,3,6 tri-o-benzoyl- | | | | |
| b-CD 3:1, BA/EA 21/79 | | | | |
| 132-144-2:BTZ+ 45-5 PLA- | 10 | 20% | 1.30 | 162 |
| PEG and 2,3,6 tri-o-benzoyl- | | | | |
| b-CD 3:1, BA/EA 21/79 | | | | |
| 132-144-3:BTZ+ 45-5 PLA- | 10 | 20% | 1.23 | 167 |
| PEG and tri-acetyl-b-CD 3:1, | | | | |
| BA/EA 21/79 | | | | |
| 132-144-4:BTZ+ 16-5 PLA- | 10 | 20% | 1.78 | 131 |
| PEG and 2,3,6 tri-o-benzoyl- | | | | |
| b-CD 3:1, BA/EA 21/79 | | | | |
| 132-144-5:BTZ+ 16-5 PLA- | 10 | 20% | 3.12 | 135 |
| PEG and triacetyl-b-CD 3:1, | | | | |
| BA/EA 21/79 | | | | |

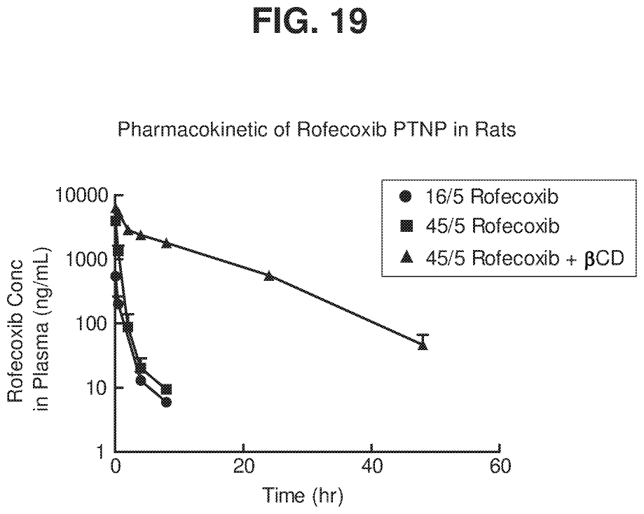

The results of the in vitro release for these formulations (FIG. 18) show that the 2,3,6 tri-o-benzoyl-β-CD formulations have a slower Bortezomib release profile than those made using triacetyl-β-CD for both the 16-5 and 45-5 PLA-PEG polymers. The formulation made with 2,3,6 tri-o-benzoyl-β-CD and 16-5 PLA-PEG has a Bortezomib release that is just as slow as the 45-5 PLA-PEG with 2,3,6 tri-o-benzoyl-β-CD formulation and considerably slower than 16-5 PLA-PEG particles alone.

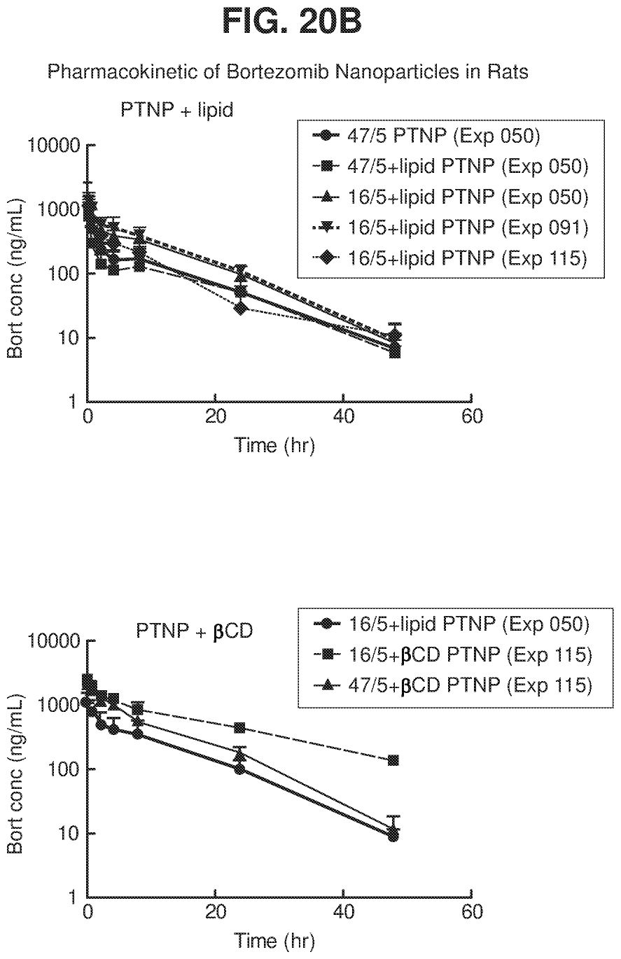

Two 2,3,6 tri-o-benzoyl-β-CD formulations with 16-5 and 45-5 PLA-PEG were remade to test for reproducibility (132-156-series). In this run the loading of the particles was about 50% lower than previous runs but the in vitro release was nearly identical. The run data can be found in Table Y. Pharmokinetics are shown in FIGS. 20A and 20B.

TABLE Y
Loading and size data of the 132-156 series
with Bortezomib and 2,3,6 tri-o-benzoyl-β-CD

| | Drug | | | |
|---|---|---|---|---|
| | theoretical | Solid | | size |
| Lot # | loading | con | Loading % | (nm) |
| 132-156-1:BTZ+ 45-5 PLA- | 10 | 20% | 0.64 | 159 |
| PEG and 2,3,6 tri-o- | | | | |
| benzoyl-b-CD 3:1 | | | | |
| 132-156-2:BTZ+ 16-5 PLA- | 10 | 20% | 1.06 | 129 |
| PEG and 2,3,6 tri-o- | | | | |
| benzoyl-b-CD 3:1 | | | | |

### Example 24

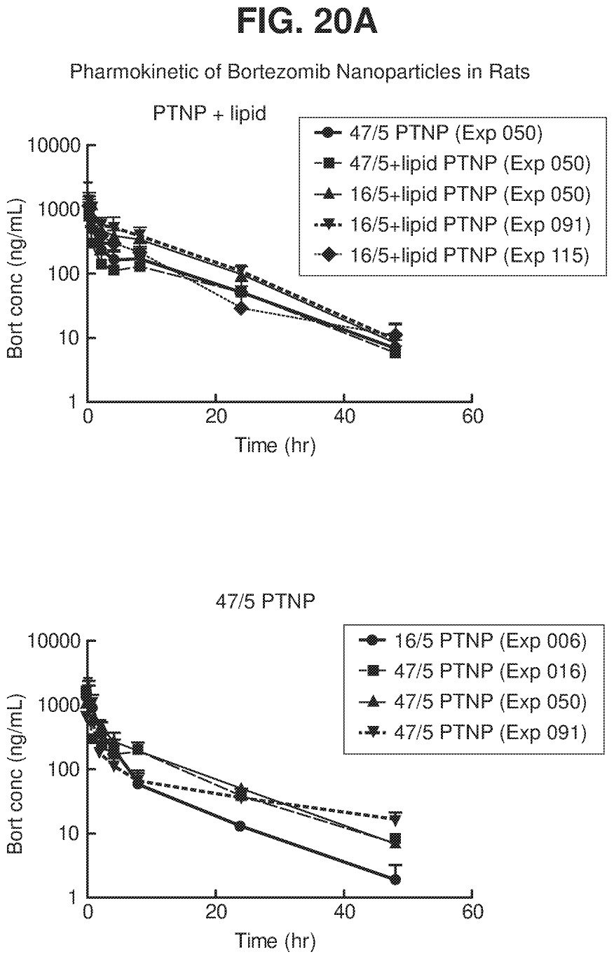

Following Example 23, nanoparticles were prepared using rofecoxib and 16/5 PLA/PEG, 50/5 PLA/PEG, 65/5 PLA/PEG, and 65/5 PLA/PEG with 80kDa PLA polymer. These nanoparticles generally have fast drug release which may be due to the small molecular weight of rofecoxib i.e. 314.36. Based on this study it was found that the addition of Heptakis(2,3,6-tri-O-benzoyl)-β-cyclodextrin considerably slowed down the drug's release and showed very favorable PK results when compared to either the 16/5 or 45/5 PLA-PEG formulations (FIG. 19).

### EQUIVALENTS

While specific embodiments of the subject invention have been discussed, the above specification is illustrative and not restrictive. Many variations of the invention will become apparent to those skilled in the art upon review of this specification. The full scope of the invention should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present invention.

### INCORPORATION BY REFERENCE

The entire contents of all patents, published patent applications, websites, and other references cited herein are hereby expressly incorporated herein in their entireties by reference.

What is claimed is:

1. A biocompatible, therapeutic polymeric nanoparticle comprising:
bortezomib or a bortezomib ester;
a biodegradable polymer comprising polylactic acid, polylactic-co-polyglycolic acid or polycaprolactone; and
optionally, a lipid and/or cyclodextrin.

2. The biocompatible, therapeutic nanoparticle of claim 1, wherein the biodegradable polymer is a block copolymer comprising a polyethylene glycol portion and a block comprising a portion selected from the group consisting of a polylactic acid portion, a poly(caprolactone) portion, and a polylactic-co-polyglycolic acid portion.

3. The biocompatible, therapeutic nanoparticle of claim 2, wherein the polyethylene glycol portion has a molecular weight of about 4 kDa to about 6 kDa.

4. The biocompatible, therapeutic nanoparticle of claim 2 or 3, wherein the block copolymer comprises a polyethylene glycol portion and a polylactic acid portion.

5. The biocompatible, therapeutic nanoparticle of any one of claims 2-4, wherein the nanoparticle comprises about 80 to about 99.9 percent by weight polyethylene glycol/polylactic acid copolymer.

6. The biocompatible, therapeutic nanoparticle of any one of claims 1-5, further comprising a homopolymer selected from the group consisting of polylactic acid homopolymer, polylactic-co-polyglycolic acid homopolymer, and poly(caprolactone) homopolymer.

7. The biocompatible, therapeutic nanoparticle of claim 6, wherein the homopolymer is a poly(lactic) acid homopolymer.

8. The biocompatible, therapeutic nanoparticle of claim 7, wherein the poly(lactic) acid homopolymer has an amine end group and a carboxylic end group and/or the poly (lactic) acid homopolymer has a weight average molecular weight of about 2,000 to about 130,000.

9. The biocompatible, therapeutic nanoparticle of any one of claims 6-8, wherein the therapeutic nanoparticle comprises about 40 to about 60 weight percent diblock poly(lactic)acid-poly(ethylene)glycol copolymer and about 40 to about 60 weight percent poly (lactic) acid homopolymer.

10. The biocompatible, therapeutic nanoparticle of claim 1, comprising about 93 to about 98 weight percent mPEG-/PLA and about 1 to about 6 percent by weight bortezomib, wherein the weight average molecular weight of the mPEG is about 5000 and the weight average molecular weight of the /PLA is about 16,000.

11. The biocompatible, therapeutic polymeric nanoparticle of claim 1, wherein the bortezomib ester is formed from bortezomib and a diol or a beta-hydroxy carboxylic acid

12. The biocompatible, therapeutic polymeric nanoparticle of claim 11, wherein the diol is a monoglyceride, optionally conjugated to polyethylene glycol, or wherein the beta-hydroxy carboxylic acid is pamoic acid or xinafoic acid.

13. The biocompatible therapeutic polymeric nanoparticle of claims 11, wherein the diol is selected from 1-undecanoyl-rac-glycerol, monomyristin, monolaurin, and monocaprin.

14. The biocompatible, therapeutic polymeric nanoparticle of claim 11, wherein the diol is a biocompatible polymer having a diol functionality.

15. The biocompatible, therapeutic polymeric nanoparticle of claim 14, wherein the diol comprises a polymer selected from the group consisting of poly(ethyleneglycol)-polydepsipeptide, poly (hydroxypropylmethacrylamide), and poly(methacrylic acid) ester.

16. The biocompatible, therapeutic polymeric nanoparticle of claim 11, wherein the diol is selected from the group consisting of 1,2-propanediol, 1,3-propanediol, 1,2-butanediol, 1,3-butanediol, 2,3-butanediol, pinanediol, pinacol, perfluoropinacol, catechol, and 1,2-cyclohexanediol.

17. The biocompatible, therapeutic polymeric nanoparticle of claim 1, wherein the bortezomib ester is represented by:
wherein
Z is
or Z₁;
Q is a biocompatible polymer, a poly(ethylene) glycol conjugated lipid, or C₅-C₁₅alkyl;
Z₁ is selected independently for each occurrence, from H and C₁-C₅ alkyl;
Y is a bond or (CH₂)ₙ, where n is 1 or 2;
and
R′ is H or C₁-C₃alkyl.

18. The biocompatible, therapeutic polymeric nanoparticle of claim 17, wherein Q is a biodegradable polymer comprising poly(methacrylate), poly(2,3-dihydroxypropyl methacrylamide), or poly(ethylene)glycol-poly(depsipeptide).

19. The biocompatible, therapeutic polymeric nanoparticle of claim 1, wherein the bortezomib ester is formed from bortezomib and dextran.

20. The biocompatible, therapeutic polymeric nanoparticle of claim 19, where the dextran is conjugated to poly(ethylene)glycol, poly(lactic) acid or poly(lactic)(glycolic) acid.

21. The biocompatible, therapeutic polymeric nanoparticle of claim 1, wherein the bortezomib ester is formed from bortezomib and poly(lactic)-acid conjugated to a mono or disaccharide.

22. The biocompatible, therapeutic polymeric nanoparticle of any one of claims 1-21, wherein the lipid is present and is a glyceride.

23. The biocompatible, therapeutic polymeric nanoparticle of claim 22, wherein the glyceride is a monoglyceride.

24. The biocompatible, therapeutic polymeric nanoparticle of claim 23, wherein the monoglyceride is lauroyl-rac-glycerol.

25. The biocompatible, therapeutic polymeric nanoparticle of any one of claims 22-24, wherein the glyceride is homogeneously dispersed within the nanoparticle.

26. A biocompatible, therapeutic polymeric nanoparticle comprising:
bortezomib;
a diblock copolymer of poly(lactic) acid and polyethylene (glycol) or a diblock copolymer of poly(lactic)-co-poly (glycolic) acid-poly(ethylene)glycol.

27. The biocompatible, therapeutic polymeric nanoparticle of claim 26, wherein the nanoparticle comprises about 0.1 to about 15 percent by weight bortezomib.

28. A biocompatible, therapeutic polymeric nanoparticle comprising:
bortezomib;
a diblock copolymer of poly(lactic) acid and polyethylene (glycol) or a diblock copolymer of poly(lactic)-co-poly (glycolic) acid-poly(ethylene)glycol; and
a glyceride.

29. The biocompatible, therapeutic polymeric nanoparticle of claim 28, wherein the glyceride is lauroyl-rac-glycerol.

30. The biocompatible, therapeutic polymeric nanoparticle of any one of claims 28-29, wherein the glyceride is homogeneously dispersed within the nanoparticle.

31. The biocompatible, therapeutic polymeric nanoparticle of any one of claims 28-30, wherein the nanoparticle comprises about 0.1 to about 35 percent by weight bortezomib.

32. The biocompatible, therapeutic polymeric nanoparticle of any one of claims 1-31, wherein the nanoparticle further comprises sodium tetraphenylborate.

33. The biocompatible, therapeutic polymeric nanoparticle of any one of claims 1-32, wherein the nanoparticle further comprises a targeting ligand.

34. A composition comprising a plurality of biocompatible, therapeutic polymeric nanoparticles of any one of claims 1-33, and a pharmaceutically acceptable excipient.

35. A method of treating a hematologic malignancy, multiple myeloma or mantle cell lymphoma comprising administering to a patient in need thereof the composition of claim 34.

36. A plurality of therapeutic nanoparticles prepared by:
combining bortezomib, a diblock copolymer of poly(lactic) acid and polyethylene (glycol) or a diblock copolymer of poly(lactic)-co-poly (glycolic) acid-poly(ethylene)glycol, and optionally a glyceride with an organic solvent to form a first organic phase having about 10 to about 40% solids;
combining the first organic phase with a first aqueous solution to form a second phase;
emulsifying the second phase to form an emulsion phase;
quenching the emulsion phase to form a quenched phase;
adding a drug solubilizer to the quenched phase to form a solubilized phase; and
filtering the solubilized phase to recover the nanoparticles, thereby forming a slurry of therapeutic nanoparticles each having about 0.1 to about 35 weight percent of bortezomib.

37. The plurality of therapeutic nanoparticles of claim 36, wherein the glyceride is lauroyl-rac-glycerol.

38. The plurality of therapeutic nanoparticles of any one of claims 36-37, wherein the glyceride is homogeneously dispersed within the nanoparticle.
